(19) United States
(12) Patent Application Publication
(10) Pub. No.: US 2005/0226893 A1
(43) Pub. Date: Oct. 13, 2005

(54) Lyophilization method to improve excipient crystallization

(76) Inventors: Jennifer Juneau, Sterling, MA (US); Susan Knowles, Melrose, MA (US)

Correspondence Address: WILMER CUTLER PICKERING HALE AND DORR LLP / WYETH, 60 STATE STREET, BOSTON, MA, 02109, US

(21) Appl. No.: 11/071,960

(22) Filed: Mar. 4, 2005

Related U.S. Application Data
(60) Provisional application No. 60/580,140, filed on Jun. 15, 2004.
(60) Provisional application No. 60/550,020, filed on Mar. 4, 2004.

(51) Int. Cl. A61K 48/00; A61K 38/16; F26B 5/06; A61K 39/12

(52) U.S. Cl. 424/204.1; 514/12; 514/44; 34/286

(57) ABSTRACT

The present invention provides improved methods to lyophilize (freeze-dry) active ingredients such as proteins, nucleic acids and viruses. The present methods improve the degree of excipient crystallization during lyophilization over prior methods. The improvement in excipient crystallization is based, in part, on a high-temperature annealing step is conducted prior to or at the same time as secondary drying. Importantly, the high-temperature annealing step does not destabilize active ingredients. Further, the high-temperature annealing step does not require sub-zero annealing steps prior to its enactment in order to provide complete excipient crystallization.

This application claims priority to U.S. Provisional Patent Application Ser. No. 60/580,140, filed on Jun. 15, 2004, and to U.S. Provisional Patent Application Ser. No. 60/550,020, filed on Mar. 4, 2004; both of which are hereby incorporated in their entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### BACKGROUND OF THE INVENTION

Due to potential instability and degradation, active ingredients, such as proteins, nucleic acids and viruses (for example, as components of vaccines) often have to be made into solid forms to achieve an acceptable shelf life as pharmaceutical products. The most commonly used method for preparing solid protein pharmaceuticals is lyophilization. Lyophilization traditionally consists of two major steps: (1) freezing of a protein solution, and (2) drying of the frozen solid under vacuum. The drying step is further divided into two phases: primary and secondary drying. The primary drying step attempts to remove the frozen water or solvent (sublimation) and the second drying step attempts to remove the non-frozen ‘bound’ water or solvent (desorption). The removal of water or other solvent by lyophilization stabilizes pharmaceutical formulations by greatly reducing the degradation rate of the active ingredient. The process inhibits the degradation process by removing solvent components in a formulation to levels that no longer support chemical reactions or biological growth. Additionally, the removal of solvent reduces molecular mobility, reducing the potential for degradative reaction. The removal of solvents is accomplished, first, by freezing the formulation such that the freezing process separates the solvent or solvents from solutes and immobilizes any non-frozen solvent molecule in the interstitial regions between the frozen solvent crystals. The solvent is then removed by sublimation (primary drying) and next by desorption (secondary drying).

The solutes in the solution prior to lyophilization comprise the protein or drug of interest (active ingredient) and the inactive ingredients (excipients). Upon lyophilization, excipients may remain in the same phase as the protein or they may phase-separate from the protein (active ingredient)-containing phase. The protein-containing phase is typically an amorphous phase. When excipients phase-separate from the protein-containing phase, they can form a crystalline phase or an amorphous phase.

Further, crystallizing excipients are commonly used in lyophilized products as bulking agents and sometimes as stabilizers. Commonly used crystallizing excipients include amino acids such as glycine, polyols such as mannitol and salts such as sodium chloride. It is usually desirable for crystallizing excipients to be fully crystalline after lyophilization. If crystallizing excipients are not fully crystallized after lyophilization, they may remain in the same amorphous phase as the protein. This can destabilize the protein by allowing for greater molecular mobility. Complete crystallization enhances drying and cake structure, thereby reducing final residual moisture levels. Complete crystallization also prevents unwanted crystallization from occurring upon storage. Since a higher degree of crystallization reduces the amount of amorphous material, a higher glass transition temperature is achieved. Typically, crystallization of these kinds of excipients is accomplished through a low-temperature annealing step conducted at sub-zero (centigrade) temperatures prior to primary drying. However, this sub-zero annealing process is slow and does not always result in sufficient or complete crystallization. In particular, mixing of glycine and sodium chloride inhibits the crystallization of either excipient and such a low temperature annealing process is inefficient in promoting crystallization, as multiple low temperature annealing steps may be needed to further crystallize the excipient.

### SUMMARY OF THE INVENTION

The present invention provides improved methods to lyophilize (freeze-dry) active ingredients, including proteins, nucleic acids and viruses. The present methods improve the degree of excipient crystallization during lyophilization over prior methods. The improvement in excipient crystallization is based, in part, in the introduction of an annealing step prior to secondary drying. This annealing step occurs at a high temperature (above 0° C.) and does not require multiple sub-zero annealing steps prior to its enactment. Although active ingredients, such as proteins, viruses and nucleic acids are inherently thermally unstable such that exposure to high temperature causes degradation, the present invention provides the unexpected finding that high-temperature annealing does not cause the degradation or instability of active ingredients. Further, the present invention provides lyophilized products produced by the present lyophilization methods, where that the products comprise both glycine and sodium chloride, and where the glycine is substantially completely crystallized or more crystallized (or more crystalline) in respect to prior methods that do not include high-temperature annealing.

In one aspect, the present invention provides a method for lyophilizing an aqueous pharmaceutical formulation, the method comprising: (a) freezing the aqueous pharmaceutical formulation at a temperature of less than −10° C.; (b) drying the pharmaceutical formulation of step (a) at a temperature between about −35° C. and about 20° C.; (c) annealing the pharmaceutical formulation of step (b) at a temperature greater than about 25° C.; and (d) drying the pharmaceutical formulation of step (c) at a temperature less than the temperature used in step (c). In one aspect of the present invention, the temperature in step (a) is less than −35° C. and the freezing is conducted for a duration of greater than 1 hour. In another aspect, the temperature in step (b) is between about −30° C. and about 20° C., or between about −25° C. and about 10° C., or at about 0° C. In another aspect, the temperature in step (c) is between about 25° C. and about 75° C., or between about 35° C. and about 60° C., or at about 50° C. In another aspect, the temperature in step (d) is between about 25° C. and about 35° C.; in one aspect, the temperature in step (d) is at about 25° C. The aqueous pharmaceutical formulation that is lyophilized by the present methods can contain essentially any active ingredient, including, but not limited to, proteins, peptides, nucleic acids and viruses.

In another aspect, the present invention provides a method for lyophilizing an aqueous pharmaceutical formulation, the method comprising: (a) freezing the aqueous pharmaceutical formulation at a temperature of less than −10° C.; (b) annealing the pharmaceutical formulation of step (a) at a temperature between about −35° C. and about 0° C.; (c) drying the pharmaceutical formulation of step (b) at a temperature between about −35° C. and about 10° C.; (d) annealing the pharmaceutical formulation of step (c) at a temperature between about 25° C. and about 75° C.; and (e) drying the pharmaceutical formulation of step (d) at a temperature less than the temperature used in step (d). In another aspect, the temperature in step (b) is between about −25° C. and −10° C., or between about −20° C. and −10° C., or at about −15° C. In another aspect, the temperature in step (c) is between about −30° C. and 5° C., or between about −25° C. and 10° C., or between about −20° C. and 0° C., or between about −20° C. and −10° C., or at about 0° C. In another aspect, the temperature in step (d) is between about 35° C. and 60° C. or at about 50° C. In another aspect, the temperature in step (e) is about 25° C. In yet another aspect, the methods can further comprise a refreezing step that is conducted after step (b) and prior to step (c), where the refreezing step comprises freezing the formulation at a temperature of less than −35° C., or at about −40° C. to about −50° C.

In another aspect, the present invention provides lyophilization methods wherein the aqueous pharmaceutical formulation comprises at least one crystallizing excipient. The crystallizing excipient(s) may be selected from the group consisting of an amino acid, a salt and a polyol. In one aspect, the amino acid is glycine or histidine. In another aspect, the salt is sodium chloride. In another aspect, the polyol is mannitol. In one aspect, the aqueous pharmaceutical formulation comprises a combination of crystallizing excipients, wherein the combination is a salt and an amino acid. In one aspect, the salt in the combination is sodium chloride, wherein the sodium chloride is present in the formulation at a concentration greater than about 25 mM, or at a concentration between about 25 mM and 200 mM, 30 mM and 100 mM, or 40 mM and 60 mM, or at a concentration of about 50 mM. In another aspect, the amino acid in the combination is present in the formulation at a concentration between about 1% to about 10%, 1.5% to 5%, 1.5% to 3%, or at about 2%. In another aspect, the amino acid in the combination is glycine.

In one aspect, the present invention provides a method for lyophilizing an aqueous pharmaceutical formulation, wherein the method comprises: (a) freezing the aqueous pharmaceutical formulation at a temperature of less than −35° C.; (b) optionally annealing the pharmaceutical formulation of step (a) at a temperature between about −20° C. and about −10° C.; (c) drying the pharmaceutical formulation of step (b) at a temperature between about −10° C. and about 10° C.; (d) annealing the pharmaceutical formulation of step (c) at a temperature between about 35° C. and about 60° C. or between about 35° C. and about 50° C.; and (e) drying the pharmaceutical formulation of step (d) at a temperature less than the temperature used in step (d).

In one aspect, the present invention provides a method for lyophilizing an aqueous pharmaceutical formulation comprising sodium chloride and glycine, wherein the method comprises: (a) freezing the aqueous pharmaceutical formulation at a temperature of less than −35° C.; (b) optionally annealing the pharmaceutical formulation of step (a) at a temperature between about −20° C. and about −10° C.; (c) drying the pharmaceutical formulation of step (b) at a temperature between about −10° C. and about 10° C.; (d) annealing the pharmaceutical formulation of step (c) at a temperature between about 35° C. and about 50° C.; and (e) drying the pharmaceutical formulation of step (d) at a temperature less than the temperature used in step (d).

In another aspect, the present invention provides a method for lyophilizing an aqueous pharmaceutical formulation comprising greater than 35 mM sodium chloride and between about 250 mM and about 300 mM glycine or between about 250 mM and about 270 mM glycine, wherein the method comprises: (a) freezing the aqueous pharmaceutical formulation at a temperature of less than −35° C.; (b) annealing the pharmaceutical formulation of step (a) at about −15° C.; (c) drying the pharmaceutical formulation of step (b) at about 0° C.; (d) annealing the pharmaceutical formulation of step (c) at about 50° C.; and (e) drying the pharmaceutical formulation of step (d) at about 25° C. This method can further comprise a refreezing step after step (b) and prior to step (c), wherein the refreezing step comprises freezing the pharmaceutical formulation of step (b) at about −40° C. to about −50° C.

In one aspect, the present invention provides a method for increasing excipient crystallization during lyophilization comprising: (a) providing an aqueous pharmaceutical formulation comprising sodium chloride and another bulking agent such as glycine; (b) freezing the aqueous pharmaceutical formulation; (c) optionally annealing the pharmaceutical formulation of step (b) at a temperature between about −35° C. and about 0° C., or between about 20° C. and about −10° C.; (d) drying the pharmaceutical formulation of step (b) or of step (c) at a temperature between about −35° C. and about 10° C. or between about −5° C. and about 5° C.; (e) annealing the pharmaceutical formulation of step (d) at a temperature between about 25° C. and about 75° C., such that the bulking agent and/or sodium chloride is more crystallized after step (e) than before step (e); and (f) drying the pharmaceutical formulation of step (e) at a temperature that is the same or lower as the temperature used in step (e), thereby increasing excipient crystallization. In this method, the bulking agent can comprise glycine, alanine or mannitol (in addition to the sodium chloride), for example. In one aspect, the bulking agent is glycine. In another aspect, this method for increasing excipient crystallization during lyophilization further comprises a refreezing step conducted after step (c) and prior to step (d), where the refreezing step comprises freezing the formulation from step (c) at a temperature between about −40° C. and −50° C., or at a temperature of about −50° C.

In another aspect, the present invention provides a lyophilized product produced by a process comprising: (a) providing a formulation comprising glycine and sodium chloride; (b) freezing the formulation; (c) optionally annealing the formulation of step (b) at a temperature between about −35° C. and about 0° C.; (d) drying the formulation of step (c) at a temperature between about −35° C. and about 10° C.; (e) annealing the formulation of step (d) at a temperature between about 25° C. and about 70° C.; and (f) drying the formulation of step (e) at a temperature that is the same or lower as the temperature used in step (e), thereby providing the lyophilized product. The active ingredient in the formulation that is lyophilized by this process can comprise a protein, a nucleic acid or a virus. Further, the glycine in the lyophilized product after step (f) can be more crystalline than before step (e).

In one aspect, the present invention provides a lyophilized product produced by the process comprising: (a) providing a formulation comprising glycine and sodium chloride; (b) freezing the formulation; (c) optionally annealing the formulation of step (b) at a temperature between about −20° C. and about −10° C.; (d) drying the formulation of step (c) at a temperature between about −5° C. and about 5° C.; (e) annealing the formulation of step (d) at a temperature between about 35 ° C. and about 60° C. or between about 35 ° C. and about 50° C.; and (f) drying the formulation of step (e) at a temperature that is the same or lower as the temperature used in step (e), thereby providing a lyophilized product. In another aspect, the glycine in the lyophilized product after step (f) is substantially completely crystallized or more crystalline than without step (e) (or more crystalline in respect to a lyophilization method that does not comprise a high-temperature annealing step before secondary drying). In another aspect, the lyophilized product is substantially stable over long time periods at high storage or accelerated temperatures. The long time periods can be, for example, at least 1 month, 3 months, 6 months, 1 year or longer. The high storage or accelerated temperatures can be, for example, between about 25° C. and about 50° C. The stability can be tested by, for example, the percent of HMW entities present in a lyophilized product, the concentration of the active ingredient, or the activity of the active ingredient.

### BRIEF DESCRIPTION OF THE DRAWINGS

FIG. 1 depicts a representative differential scanning calorimetry (DSC) first scan (FIG. 1A) and second scan (FIG. 1B) of a solid cake of formulation 1 lyophilized according cycles Lyo G, H or I (see Table 2 for contents of formulations 1, 2 and 3; see Tables 3, 4 and 5 for steps of cycles of Lyo G, H and I; see Example 1 for description of experiments). A crystallization event was observed in the first scans of the solid cakes of formulation 1 lyophilized according to cycles Lyo G, H and I, which indicates a lack of complete or substantial crystallization in the cakes. As stated within, when a DSC first scan shows crystallization, this indicates that complete or substantially complete crystallization did not occur in a lyophilized cake. The first scan thus shows that a crystallization event occurred, and the second scan confirms that an exothermic event was recrystallization.

FIG. 2 depicts a representative DSC first scan (FIG. 2A) and second scan (FIG. 2B) of a solid cake of formulation 2 lyophilized according cycles Lyo G, H or I (see Table 2 for contents of formulations 1, 2 and 3; see Tables 3, 4 and 5 for steps of cycles of Lyo G, H and I; see Example 1 for description of experiments). A crystallization event was observed in the first scans of the solid cakes of formulation 2 lyophilized according to cycles Lyo G, H and I, which indicates a lack of complete or substantial crystallization in the cakes. The second scans confirm that the exothermic event observed in the first scans is crystallization. Further, the second scans did not show a transition T_{g}.

FIG. 3 depicts a representative DSC first scan (FIG. 3A) and second scan (FIG. 3B) of a solid cake of formulation 3 lyophilized according cycles Lyo G, H or I (see Table 2 for contents of formulations 1, 2 and 3; see Tables 3, 4 and 5 for steps of cycles of Lyo G, H and I; see Example 1 for description of experiments). A crystallization event was observed in the first scans of the solid cakes of formulation 3 lyophilized according to cycles Lyo G, H and I, which indicates a lack of complete or substantial crystallization in the cakes. The second scans confirm that the exothermic event observed in the first scans is crystallization. Further, the second scans did not show a transition T_{g}.

FIG. 4 depicts a DSC first scan (FIG. 4A) and second scan (FIG. 4B) of a solid cake of formulation 4 (“fix927lyoJ.001”; the long dash- dot graph line), formulation 5 (“fix50250lyoja.001”; non-dashed line) and formulation 6 (“fix50270lyoja.001”; dashed line), lyophilized according to cycle Lyo J (see Table 7 for contents of formulations 4, 5 and 6; see Tables 8, 9, and 10 for steps of cycles of Lyo J, K, and L; see Example 2 for description of experiments). Table 11 summarizes the DSC first and second scan data in Example 2, where formulations 5 and 6 lyophilized according to Lyo J showed crystallization in the first scan, and no transition T_{g} in the second scan.

FIG. 5 depicts a DSC first scan (FIG. 5A) and second scan (FIG. 5B) of a solid cake of formulation 4 (“fix927yoK.002”; long dash-dot graph-line), formulation 5 (“fix50250yok.001”; non-dashed line) and formulation 6 (“fix50270yok.001”; dashed line), lyophilized according to cycle Lyo K (see Table 7 for contents of formulations 4, 5 and 6; see Tables 8, 9, and 10 for steps of cycles of Lyo J, K, and L; see Example 2 for description of experiments). FIG. 5C presents data from another second scan on the solid cake of formulation 4 (“fix 927 lyo K”) lyophilized by the Lyo K cycle. Table 11 summarizes the DSC first and second scan data in Example 2, where formulations 4, 5 and 6 lyophilized according to Lyo K did not show crystallization in the first scan, and had a transition T_{g} in the second scan.

FIG. 6 depicts a DSC first scan (FIG. 6A) and second scan (FIG. 6B) of a solid cake of formulation 4 (“fix927lyol.001”; long dash-dot graph-line), formulation 5 (“fix50250lyoL.001”; non-dashed line) and formulation 6 (“fix50270lyoL.001”; dashed line), lyophilized according to cycle Lyo L (see Table 7 for contents of formulations 4, 5 and 6; see Tables 8, 9, and 10 for steps of cycles of Lyo J, K, and L; see Example 2 for description of experiments). FIG. 6C presents data from another second scan on the solid cake of formulation 4 (“fix 927 lyo L”) lyophilized by the Lyo L cycle. Table 11 summarizes the DSC first and second scan data in Example 2, where formulations 4, 5 and 6 lyophilized according to Lyo L did not show crystallization in the first scan, and had a transition T_{g} in the second scan.

FIG. 7 presents the percent of high molecular weight species present in the pre-lyophilized formulations and post-lyophilized cakes of formulations 4, 5 and 6 lyophilized according to Lyo J, K and L. (See Example 2). For each formulation, 10 vials were tested and assayed in triplicate. “PCTRL” denotes formulation 4 prior to lyophilization; “CTRL” denotes formulation 4 after lyophilization. “P50/250” denotes formulation 5 prior to lyophilization; “50/250” denotes formulation 5 after lyophilization. “P50/270” denotes formulation 6 prior to lyophilization; “50/270” denotes formulation 6 after lyophilization.

FIG. 8 presents the clotting activity (FIG. 8A) and the percent recovery of clotting activity (FIG. 8B) of the Factor IX protein in the pre-lyophilized formulations and post-lyophilized cakes of formulations 4, 5 and 6 lyophilized according to Lyo J, K and L. (See Example 2). For each formulation, eight vials per post-lyophilization formulation were tested. Due to a 4 mL fill and a 5 mL reconstitution dilution, 80% recovery is the highest recovery value expected in FIG. 8B.

FIG. 9 presents the percent recovery of specific activity of the Factor IX protein in the pre-lyophilized formulations and post-lyophilized cakes of formulations 4, 5 and 6 lyophilized according to Lyo J, K and L. (See Example 2).

FIG. 10 presents x-ray diffraction (XRD) patterns on the cake of formulation 2 lyophilized according to Lyo G (FIG. 10A) and on the cake of formulation 6 lyophilized according to Lyo L (FIG. 10B). The XRD patterns indicate that crystalline glycine is present in the lyophilized samples. Further, the XRD patterns show a qualitative pattern that a glycine/sodium chloride formulation lyophilized by Lyo L has more crystalline material than a glycine/sodium chloride formulation lyophilized by Lyo G. The XRD peak heights are relative to crystallinity, where higher peaks reflect the presence of more crystalline material.

FIG. 11 shows an XRD pattern on the cake of formulation 7 lyophilized according to Lyo Q (see Example 3). The XRD pattern for Lyo Q is compared to an XRD pattern for Lyo G. Lyo G was used for comparison because in includes the same lyophilization cycle parameters as Lyo Q except that Lyo G does not have a 50° C. thermal treatment. The XRD pattern comparison shows that lyophilization by Lyo Q results in an increase in glycine crystallization, as can be observed by the intensity of the 17.5° peak.

### DETAILED DESCRIPTION OF THE INVENTION

The present invention relates to improved processes for lyophilizing pharmaceutical formulations. Lyophilization is important, in part, because it helps to stabilize active ingredients (such as proteins, nucleic acids and viruses) by slowing or preventing degradation. The present methods provide improved excipient crystallization during lyophilization steps such that stability and efficiency of lyophilized products are thereby improved. It is usually desirable for crystallizing excipients to be fully crystalline after lyophilization because if crystallizing excipients are left in the protein- or drug-containing amorphous phase, the excipients reduce the glass transition temperature (T_{g}) of that phase. An amorphous phase with reduced T_{g} may have increased molecular mobility. Increased molecular mobility can allow for increased rates of degrading reactions. Thus, for a drug that is expected to be in the amorphous phase, raising the glass transition temperature of that phase will result in enhanced stability. Conversely, a formulation with a depressed glass transition temperature due to uncrystallized excipient still left in the amorphous phase because of poor lyophilization methods, would be expected to have poorer stability.

Prior methods crystallized excipients through an annealing step(s) conducted at sub-zero centigrade temperatures prior to primary drying. However, this sub-zero annealing process is slow and does not always result in complete crystallization. In particular, mixing of glycine and sodium chloride inhibits the crystallization of either excipient, and such a low temperature annealing process is inefficient in enhancing excipient crystallization as longer cycles would be required. In contrast, the present invention provides methods that introduce a high-temperature annealing step prior to secondary drying such that multiple sub-zero annealing steps are not required for enhanced excipient crystallization. Further, although active ingredients, such as proteins, viruses and nucleic acids are inherently thermally unstable such that exposure to high temperature causes degradation, the present invention provides the unexpected finding that high-temperature annealing does not cause the degradation or instability of active ingredients. Thus, the present invention provides efficient methods to enhance excipient crystallization during lyophilization, including enhancing excipient crystallization of formulations comprising both glycine and sodium chloride.

As used herein, the terms “lyophilization”, “lyophilized” and “freeze-dried” relate to processes such as “freezing” a solution followed by “drying.” In general, the lyophilization methods of the present invention comprise the following steps: (1) freezing, (2) primary drying, (3) high-temperature annealing at a temperature greater than about 25° C., and (4) secondary drying at a temperature that is the same or lower than the temperature at the high-temperature annealing step. One or more optional low-temperature annealing steps can be conducted before primary drying, and an optional refreezing step can be conducted after the low-temperature annealing step.

The present invention provides the unexpected finding that a high-temperature annealing step prior to secondary drying does not destabilize the active ingredient, and thus the present methods are able to improve excipient crystallization while providing a more efficient, practical or robust lyophilization protocol. The present lyophilization methods allow for an increased degree of crystalline bulking agents over prior methods, while maintaining the stability and activity of the active ingredient. The present invention sometimes refers to the objective of complete excipient crystallization, and one skilled in the art understands that “complete crystallization” is difficult to verify, because the current sensitivity of technology cannot inform one with absolute certainty that an excipient is 100% crystallized. Therefore, in practical terms, the invention provides lyophilization methods that improve excipient crystallization in respect to prior methods. Accordingly, as used herein, “complete crystallization” of lyophilized products can be assessed, for example, by differential scanning calorimetry (DSC), where one skilled in the art recognizes that a non-reversible exothermic event on a first scan represents a crystallization event, which indicates that a crystallizing excipient did not completely crystallize during lyophilization (see Examples).

### Lyophilization Processes

Lyophilization cycles traditionally include three phases: freezing (thermal treatment), primary drying (sublimation) and secondary drying (desorption). In various embodiments, the present invention improves on traditional lyophilization processes by introducing one or more annealing phases prior to secondary drying, where the annealing and drying phases occur at specific temperature ranges. In the present invention, specific temperatures and temperature ranges of a lyophilization process refer to the shelf temperature of the lyophilizer equipment, unless otherwise noted. The shelf temperature refers to the control temperature for coolant flowing through the shelves of the lyophilizer, which is what one controls in terms of temperature during lyophilization. The temperature of the sample (the product temperature) depends on the shelf temperature, the chamber pressure and the rate of evaporation/sublimation during primary drying (evaporative cooling makes product temperatures less than the shelf temperature). The present invention provides improved lyophilization processes in order to provide, for example, a more consistent, stable and aesthetically acceptable product.

In the present invention, percentages are weight/weight when referring to solids and weight/volume when referring to liquids.

Formulaic Terms:

Ice (solvent crystal) formation during cooling of a pharmaceutical formulation concentrates all solutes. Solute concentration eventually changes the solution from a liquid to a glass. The temperature of this reversible transition for the freeze-concentrated solution is termed the glass transition temperature, T_{g}′, of the maximally freeze-concentrated solution. This temperature is also called the temperature of vitreous transformation. T_{g}′ is used to differentiate this transition from the softening point of a true glass transition, T_{g}, of a pure polymer. The collapse temperature, T_{col} is the temperature at which the interstitial water in the frozen matrix becomes significantly mobile. For reference, Table 1 lists some commonly used excipients and buffers (and proteins, where these proteins are not the active ingredient of a formulation, but rather are additional elements to a formulation).

TABLE 1
List of Buffers, Excipients and Proteins
Compound

Buffering Agents: citric acid; Hepes; histidine; potassium acetate; potassium citrate;
potassium phosphate; sodium acetate; sodium bicarbonate; sodium citrate; Tris
(tromethamine)-base; Tris-HCl.
Excipients, low MW: β-alanine; arabinose; arginine; cellobiose; fructose; fucose; galactose;
glucose; glutamic acid; glycerol; glycine; histidine; lactose; lysine; maltose; maltotriose;
mannitol; mannose; melibiose; octulose; raffinose; ribose; sodium chloride; sorbitol
(glucitol); sucrose; trehalose; water; xylitol; xylose. (One skilled in the art understands that
some of these excipients are crystallizing excipients, such as glycine, mannitol and sodium
chloride; and some of these excipients form amorphous phases).
Excipients, high MW: cellulose; β-cyclodextrin; dextran; ficoll; gelatin;
hydroxypropylmethyl-cellulose; hydroxyethyl starch; maltodextrin 860; methocel; PEG;
polydextrose; PVP; sephadex; starch
Proteins: BSA; α-casein, globulins; HSA; α-lactalbumin; LDH, lysozyme; myoglobin;
ovalbumin; RNase A.

When the temperature of an aqueous formulation drops below 0° C., water usually crystallizes out first. Then depending on the freezing rate, crystallizable components that have the least solubility in the formulation may crystallize next. This temperature (the temperature at which the crystallizable components in a formulation crystallize) is termed the crystallization temperature. When the temperature of an aqueous formulation further decreases after crystallization of the least soluble component, the crystallizable component(s) and water crystallize out at the same time as a mixture. This temperature is termed the eutectic crystallization/melting temperature, Tₑᵤₜ. Due to excipient interactions, some multicomponent formulations do not exhibit Tₑᵤₜ.

Freezing:

The first step in lyophilization is the freezing step. The formulation or sample is frozen solid, which converts the water content of the material to ice. In one embodiment, freezing an aqueous pharmaceutical formulation can be conducted at a temperature of less than −10° C. In another embodiment, freezing can be conducted at or below −35 or −50° C. In the present invention, once the freezing temperature (as in the shelf temperature) reaches a target temperature of between about −35° C. to about −50° C., for example, then the freezing temperature is held (a “freezing hold” step) until the sample is frozen, or for about 1 hour to about 24 hours, for about 3 hours to about 12 hours, for about 5 hours to about 10 hours, or for 5 about hours. The time of freezing depends on factors such as the volume of the solution per vial, independent of the formulation composition.

Low Temperature Annealing (an Optional Step):

In the present invention, a low-temperature annealing step is optional. Prior methods used one or more low-temperature annealing steps prior to drying because a crystallizable component may not be completely or sufficiently crystallized. However, these prior methods were inefficient as their low-temperature annealing steps required long or multiple cycles, and these prior methods are insufficient to promote sufficient or complete crystallization.

Complete crystallization of a formulation component may provide a necessary cake structure or an active ingredient may be more stable in a formulation with complete crystallization. The removal from the amorphous phase of a crystallizable component, such as glycine, can increase the T_{g}′ of the amorphous phase. The increased T_{g}′ can allow more efficient primary drying at a higher temperature. Further, more complete crystallization of an excipient can also increase T_{g} after lyophilization, which is critical for stability of the active ingredient.

In the present invention, a low-temperature annealing step can be conducted at a temperature of about −35° C. to about 0° C., or between about −25° C. and −10° C., or between about −20° C. and −10° C. In one embodiment, the low-temperature annealing step is conducted at a temperature of about −15° C.

The low-temperature annealing step temperature is attained by increasing the temperature from the freezing step (also called “freezing hold”). The process of regulating the increase of temperature from the freezing temperature to the low-temperature annealing temperature is called an “annealing ramp” step, which is optional in the present invention. The annealing ramp step can be conducted at different rates, for example, at about 0.1° C. to about 5° C. per minute.

Refreezing (an Optional Step):

The lyophilization methods of the present invention also encompass the option of including a refreezing step after the low-temperature annealing step. The refreezing step can be conducted at a freezing temperature (freezing hold temperature) of between about −35° C. to about −50° C. for about 1-10 hours, 3-7 hours or 5 hours. The refreezing ramp step can be conducted at a rate of about −0.5° C. to −5° C. per minute, for example.

Vacuum Initiation:

Directly prior to primary drying, the formulation is placed under vacuum at the temperature of the step directly prior to primary drying. This step is called “vacuum initiation.” Thus, for example, if a refreezing step is prior to primary drying, then vacuum initiation occurs at the refreezing step temperature. The vacuum can be at a level of between about 20 to about 300 microns. Once the vacuum is initiated, a vacuum is present for the rest of the lyophilization process, although the vacuum level can change.

Primary Drying:

Drying is divided into two phases: primary and secondary drying. The primary drying removes the frozen water (sublimation of ice) and the secondary drying removes non-frozen ‘bound’ water (desorption of water). In primary drying, the objective is to remove the unbound, or easily removed ice from the sample. The unbound water at the beginning of the primary drying step should be in the form of free ice, which is removed by converting it directly from a solid to a vapor, where this conversion process is called sublimation.

In the present invention, the primary drying step can be conducted at a temperature between about −35° C. and about 20° C., or between about −25° C. and 10° C., or between about −20° C. and 0° C. In one embodiment, the primary drying step is conducted at 0° C.

The regulation of the increase of temperature from the step prior to primary drying to the primary drying temperature is called the “primary drying ramp” step, which is an optional step. The primary drying ramp step can be conducted at a rate of about 0.1° C. to about 5° C. per minute.

The primary drying step can be conducted for a time sufficient to ensure that substantially all of the frozen water is removed from the sample. One skilled in the art understands that the primary drying time varies with configuration, in that the duration of primary drying depends on the fill volume and geometry (surface area of the cake—resistance/flux). In one embodiment, the duration of primary drying is greater than 10 hours, in another embodiment it is about 10 to about 100 hours. In another embodiment, the duration of primary drying is about 30 to 50 hours. In another embodiment, the duration of primary drying is 38 hours.

Several methods can be used to monitor the completion of the primary drying step. One method is to observe the changes in product temperature during freeze-drying. Another method is to observe the changes in chamber pressure, where when sublimation ends, no more water molecules are in the chamber contributing to changes in pressure. The end of the primary drying step is when the product (sample) temperature approaches the shelf-temperature, evidenced by a significant change in the slope of the product temperature trace due to a reduced sublimation rate; when sublimation ends, evaporative cooling ends. To prevent a premature ending, an extra 2 to 3 hours of primary drying may be added to the duration. Another method to monitor the completion of primary drying is the pressure rise test. By disconnecting the vacuum source, the chamber pressure should rise at a rate depending on the amount of moisture in the product. The end of the primary drying process could be set as when the rate of pressure rise is below a specified value. Another method for determining the end of the primary drying step is the measurement of the heat transfer rate (Jennings, T. A., Duan, N. (1995), J. Parent. Sci. Technol., 49, 272-282).

High-temperature Annealing:

The methods of the present invention include one or more high-temperature annealing (or thermal treatment) steps prior to secondary drying. Prior methods report that sub-zero annealing steps are necessary prior to primary drying when secondary drying is conducted at high temperatures. However, the present invention discloses methods that do not require sub-zero annealing steps in order to perform high temperature secondary drying. The present invention has determined that sub-zero annealing steps prior to high temperature drying is not necessary if a high-temperature annealing step is performed during or directly prior to secondary drying. The invention has determined that the high-temperature annealing step enhances excipient crystallization, including glycine crystallization, while maintaining active ingredient stability.

Thus, the present invention provides a high-temperature annealing step prior to secondary drying, where the high-temperature annealing step is conducted at a temperature greater than about 25° C. In one embodiment, the high-temperature annealing step temperature is about 25° C. to about 75° C. or about 35° C. to about 60° C. In another embodiment, the high-temperature annealing step is conducted at a temperature of about 50° C. In other embodiments, the high temperature annealing step is conducted at a temperature of about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60° C. In another embodiment, the temperature of the annealing step is at or above the temperature observed by differential scanning calorimetry to correspond to the on-set of the crystallization event. One skilled in the art might contemplate using a temperature slightly lower than the crystallization on-set temperature recognizing that the kinetics of crystallization may be slower and the step duration may be longer. Temperatures greater than the crystallization on-set temperature are preferred because the kinetics of crystallization are faster and the step duration may be shorter.

The regulation of the increase of temperature from primary drying to high-temperature annealing is called the “high-temperature annealing ramp” (ramp steps are implicit in the present invention, as changes from one temperature hold to another temperature hold inherently include some sort of ramping) and can be conducted at a rate of about 0.1 to about 20° C. per minute.

The duration of the high-temperature annealing step depends on many factors, including fill volume. The high-temperature annealing step can be conducted, for example, for 1 hour to about 24 hours. In one embodiment, the high-temperature annealing step is conducted for about 1 hour to about 15 hours. In another embodiment, the high-temperature step is conducted for about 10 hours. Surprisingly, the high-temperature annealing step of the invention does not negatively affect protein stability or activity (see Example 2). This is unexpected because proteins are known to be thermally unstable. Further, Example 2 shows that the high-temperature annealing step causes an increase in excipient (in this Example, glycine) crystallization.

Secondary Drying:

Even if all the free ice is removed by the aforementioned sublimation process, the sample may still contain enough bound water to limit its structural integrity and shelf life. During secondary drying, the water that is bound to the solids in the product is converted into vapor. This can be a slow process as the remaining bound water has a lower pressure than free liquid at the same temperature. Although some bound water is removed during the prior drying and annealing methods, secondary drying is required after the removal of free ice to achieve low enough residual moisture levels that provide the desired biological and structural characteristics of the final product.

Depending on the lyophilization process, mannitol can crystallize as mannitol hydrate. Upon storage, mannitol hydrate can convert to crystalline mannitol, releasing water. The released water can then (1) participate in chemical reactions and (2) lower the T_{g} of the amorphous phase, allowing for more molecular mobility and degradation reactions. The high-temperature annealing step can be used to convert crystalline mannitol hydrate to crystalline mannitol, so that the remaining water can be removed during secondary drying.

In the present invention, the secondary drying step can be conducted at a temperature that is the same or lower as the temperature used in the high-temperature step. In one embodiment, the secondary drying step is conducted at a temperature of about 0° C. to less than 35° C. or at about 15° C. to about 35° C. In another embodiment, the secondary drying step is conducted at about 25° C.

The step of regulating the decrease of the temperature from the high-temperature annealing step to the secondary drying step is called the “secondary drying ramp,” which is an optional step in the present invention. The secondary drying ramp step can be conducted at a rate of temperature decrease of about 0.1 ° C. to about 10° C. per minute.

The secondary drying step can be conducted for a time sufficient to reduce the residual moisture level in the lyophilized product to a desired level. In the invention, a desired residual moisture level is less than 2%. In one embodiment, the residual moisture level of the lyophilized product produced by the methods is less than 1%, 0.75%, 0.5%, 0.25% or 0.10%. To determine the residual moisture level in the sample, the Karl Fischer method can be used. Further, the pressure rise test or the measurement of the heat transfer rate can also be used to determine the end of the secondary drying step. Alternatively, an electronic hygrometer or a residual gas analyzer may be used (Nail, S. L., Johnson, W., (1992) Dev. Biol. Stand. 74, 137-150). Also, the minimum duration of secondary drying can be determined systematically by using different combinations of shelf temperatures (where the shelf temperature of the secondary drying step is the same or less than the temperature used in the high-temperature step) and durations. Residual moisture content of lyophilized formulations can be determined by several methods, including loss-on-drying, Karl Fischer titration, thermal gravimetric analysis (TGA), gas chromatography (GC), or infrared spectroscopy.

### Lyophilization Formulations

A formulation to be lyophilized comprises three basic components: (1) an active ingredient(s), (2) an excipient(s) and (3) a solvent(s). Excipients include pharmaceutically acceptable reagents to provide good lyophilized cake properties (bulking agents) as well as to provide lyoprotection and/or cryoprotection of proteins (“stabilizer”), maintenance of pH (buffering agents), and proper conformation of the protein during storage so that substantial retention of biological activity (including active ingredient stability, such as protein stability) is maintained. Thus, with relation to excipients, an example of a formulation may include one or more of a buffering agent(s), a bulking agent(s), a protein stabilizer(s) and an antimicrobial(s). The active ingredient, for example, refers to a reagent or a therapeutic drug. Where the active ingredient refers to a drug, the activity of the drug relates to its potency. Where the active ingredient refers to a reagent, the activity of the reagent refers to its reactivity.

Sugars/Polyols:

Many sugars or polyols are used as nonspecific protein stabilizers in solution and during freeze-thawing and freeze-drying. The level of stabilization afforded by sugars or polyols generally depends on their concentrations. In one embodiment, the present invention contemplates the use of disaccharides in formulations to be lyophilized by the disclosed methods. Disaccharides may include, but are not limited to, trehalose, sucrose, maltose, and lactose. Other sugars or polyols that may be used, include but are not limited to, glycerol, xylitol, sorbitol, mannitol, glucose, inositol, raffinose and maltotriose. Mannitol is a crystallizing polyol that can also be used as a bulking agent.

Polymers:

Polymers can be used to stabilize proteins in solution and during freeze-thawing and freeze-drying. One popular polymer is serum albumin, which has been used both as a cryoprotectant and lyoprotectant. However, the concern about blood-borne pathogens limits the application of serum albumin in therapeutic and therapeutic-related products. Thus, in one embodiment, the invention provides formulations that are albumin free which are lyophilized by the disclosed methods. Other polymers include, but are not limited to, dextran, poly(vinyl alcohol) (PVA), hydroxypropyl methylcellulose (HPMC), gelatin, polyethylene glycol (PEG) and polyvinylpyrrolidone (PVP). Polymers are not crystallizing excipients, as they form amorphous phases.

Non-aqueous Solvents:

Non-aqueous solvents generally destabilize proteins in solution. At low concentrations certain non-aqueous solvents may have a stabilizing effect. These stabilizing non-aqueous solvents include polyhydric alcohols such as PEGs, ethylene glycol, glycerol, and some polar and aprotic solvents such as dimethylsulphoxide (DMSO) and dimethylformamide (DMF). However, non-aqueous solvents are not preferred to be used with the present invention.

Surfactants:

The formation of ice-water interfaces during freezing may cause surface denaturation of proteins. Surfactants may drop surface tension of protein solutions and reduce the driving force of protein adsorption and/or aggregation at these interfaces. Also, surfactants may compete with an active ingredient for the ice/water interface during lyophilization. Surfactants can include, for example, Tween 80™ (polysorbate 80; other polysorbates are also contemplated), Brij® 35, Brij 30®, Lubrol-px™, Triton X-10®, Pluronico® F127 and sodium dodecyl sulfate (SDS).

Salts as Bulking Agents:

Various salts can be used as bulking agents. Exemplary salt bulking agents include, for example, NaCl, MgCl₂ and CaCl₂.

Amino Acids:

Certain amino acids can be used as cryoprotectants and/or lyoprotectants and/or bulking agents. Amino acids that may be used include, but are not limited to, glycine, proline, 4-hydroxyproline, L-serine, sodium glutamate, alanine, arginine and lysine hydrochloride. Short chains of amino acids, such as di- or tri-amino acids, including dilysine, may also be used. Most amino acids are potential bulking agents as they generally crystallize out easily. However, formation of acid salts reduces their tendency to crystallize. Further, an amorphous excipient(s) in a protein formulation may inhibit crystallization of the bulking agent(s), thus affecting protein stability. Thus, in prior methods, the combination of glycine and NaCl was not preferable, as NaCl has a low eutectic and glass transition temperature. In the present invention, the methods provide a high-temperature annealing step and a high temperature secondary drying step without prior sub-zero annealing steps that increases the degree of crystalline glycine even when formulated in the presence of sodium chloride.

Buffering Agents:

Many buffering agents covering a wide pH range are available for selection in formulations. Buffering agents include, for example, acetate, citrate, glycine, histidine, phosphate (sodium or potassium), diethanolamine and Tris. Buffering agents encompasses those agents which maintain the solution pH in an acceptable range prior to lyophilization.

The upper concentration limits are generally higher for “bulk” protein than for “dosage” protein forms. For example, while buffer concentrations can range from several millimolar up to the upper limit of their solubility, e.g., histidine could be as high as 200 mM, one skilled in the art would also take into consideration achieving/maintaining an appropriate physiologically suitable concentration.

Active Ingredient:

The formulations lyophilized by the present methods can include essentially any active ingredient, such as proteins, nucleic acids, viruses, and combinations thereof. Proteins can include, for example, clotting factors, growth factors, cytokines, antibodies and chimeric constructs. In relation to proteins, the active ingredients present in a formulation can be recombinant proteins or proteins isolated from an organism.

Glycine/NaCl Formulations:

With prior methods of lyophilization, formulations that comprise about 20 mM or greater of sodium chloride in the presence of glycine (typically about 2%, or about 250mM), the crystallization of glycine is inhibited by the sodium chloride. In formulations that comprise greater than 3 mM NaCl, glycine crystallization is significantly diminished when using prior methods of lyophilization. In contrast, the present invention provides lyophilization methods where salts do not substantially inhibit the crystallization of an amino acid excipient (for example, sodium chloride and glycine).

Factor IX Formulations:

In one embodiment, the present invention provides lyophilized Factor IX products made by the disclosed processes. Suitable Factor IX formulations that can be lyophilized by the present methods include Factor IX formulations disclosed in U.S. Pat. No. 6,372,716; U.S. Pat. No. 5,770,700 and U.S. Patent Application Publication No. US2001/0031721.

For example, a Factor IX formulation that can be lyophilized comprises Factor IX, a bulking agent and a cryoprotectant. The Factor IX concentration can be, for example, from about 0.1 mg/mL to about 20 mg/mL (equivalent to about 20 to at least 4000 U/mL) or from about 0.4 mg/mL to about 20 mg/mL. The bulking agents for Factor IX formulations can include, for example, glycine and/or a magnesium salt, calcium salt, sodium salt or a chloride salt, wherein the concentration of the bulking agent(s) is from about 0.5 mM to about 400 mM. In one embodiment, the bulking agent is glycine, wherein the glycine concentration is from about 0.1M to about 0.3M, from about 0.2M to about 0.3M or from about 0.25M to about 0.27M. In another embodiment, the bulking agents are glycine at a concentration from about 0.25M to about 0.27M and sodium chloride at a concentration of about 50 mM. Suitable cryoprotectants for Factor IX formulations include for example, polyols, such as mannitol and sucrose at a concentration from about 0.5% to about 2%. The Factor IX formulations can further comprise a surfactant and/or detergent, such as polysorbate (e.g., Tween-80) or polyethyleneglycol (PEG), which may also serve as a cryoprotectant during the freezing step(s). The surfactant may range from about 0.005% to about 0.05%. The concentrations of the excipients can have, for example, a combined osmolality of about 250 mOsM to about 350 mOsM, or about 300 mOsM±50 mOsM, and further, may contain an appropriate buffering agent to maintain a physiologically suitable pH, e.g., in the range of about 6.0 to 8.0. Buffering agents can include, for example, histidine, sodium phosphate or potassium phosphate, with a target pH of about 6.5 to about 7.5, all at about 5 mM to about 50 mM. In one embodiment, the Factor IX formulation comprises Factor IX, 10 mM histidine, 1% sucrose, 50 mM sodium chloride, 0.005% polysorbate 80 and 250 to 270 mM glycine. The final NaCl concentration in a reconstituted lyophilized Factor IX formulation should be ≧40 mM in order to reduce red blood cell agglutination/aggregation when the Factor IX formulation is administered. Thus, in one embodiment, Factor IX formulations that are lyophilized by the present methods comprise at least 40 mM sodium chloride.

It is to be understood and expected that variations in the principles of the invention herein disclosed in an exemplary embodiment can be made by one skilled in the art and it is intended that such modifications, changes, and substitutions are included within the scope of the present invention.

The examples set forth below illustrate several embodiments of the invention. These examples are for illustrative purposes only, and are not meant to be limiting.

### EXAMPLES

### Example 1

### Lyophilization Methods without High-Temperature Annealing

Three different lyophilization cycles were conducted in order to identify a lyophilization method that increases glycine crystallization and maintains protein stability. A further benefit that was examined for included whether the lyophilized product has a residual moisture content of less than 1%. The lyophilization cycles performed in this Example are denoted as “Lyo G”, “Lyo H” and “Lyo I” as described in Tables 3, 4 and 5, respectively. These cycles did not include a high-temperature annealing step, and as a consequence, glycine crystallization was not complete or was less complete as compared to methods that include a high-temperature annealing step. Further, the cycles without a high-temperature annealing step resulted lyophilized products with residual moisture contents less than 2%.

Table 2 shows the three formulations that were used in this Example. Each formulation contained Factor IX at 250 IU/mL at pH 6.8.

TABLE 2
Formulations

| Formulation | NaCl (mM) | Glycine (mM) |
|---|---|---|
| 1 | 50 | 250 |
| 2 | 50 | 270 |
| 3 | 50 | 300 |

Tables 3, 4 and 5 show the lyophilization steps for Lyo G, H and I. All vials were stoppered under vacuum.

TABLE 3
Lyophilization Cycle “Lyo G”

| Step | Shelf Temperature | Duration |
|---|---|---|
| Freezing Hold | −50° C. | 5 hours |
| Annealing Hold | −15° C. | 5 hours |
| Re-Freezing Hold | −50° C. | 5 hours |
| Primary Drying | 0° C. | 38 hours |
| Secondary Drying | 25° C. | 9.5 hours |

TABLE 4
Lyophilization Cycle “Lyo H”

| Step | Shelf Temperature | Duration |
|---|---|---|
| Freezing Hold | −50° C. | 5 hours |
| Annealing Hold | −10° C. | 5 hours |
| Re-Freezing Hold | −50° C. | 5 hours |
| Primary Drying | 0° C. | 38 hours |
| Secondary Drying | 25° C. | 9.5 hours |

TABLE 5
Lyophilization Cycle “Lyo I”

| Step | Shelf Temperature | Duration |
|---|---|---|
| Freezing Hold | −50° C. | 5 hours |
| Annealing Hold | −15° C. | 5 hours |
| Re-Freezing Hold | −50° C. | 5 hours |
| Primary Drying | 0° C. | 38 hours |
| Secondary Drying | 35° C. | 9.5 hours |

Formulations 1, 2, and 3 were each lyophilized in duplicate according to the protocols of Lyo G, Lyo H, and Lyo I. The residual moisture content of each lyophilized product was then assessed using Karl Fisher. As can be seen in Table 6, neither Lyo G, Lyo H nor Lyo I lyophilized the formulations such that the moisture content is less than 1%.

TABLE 6
Residual Moisture Content

| Sample | Lyo G | Lyo H | Lyo I |
|---|---|---|---|
| Formulation 1 (1) | 2.08% | 3.09% | 1.58% |
| Formulation 1 (2) | 2.02% | 2.54% | 1.63% |
| Formulation 2 (1) | 2.08% | 3.22% | 1.44% |
| Formulation 2 (2) | 2.10% | 2.59% | 1.48% |
| Formulation 3 (1) | 2.03% | 2.25% | 1.17% |
| Formulation 3 (2) | 1.93% | 2.93% | 1.13% |

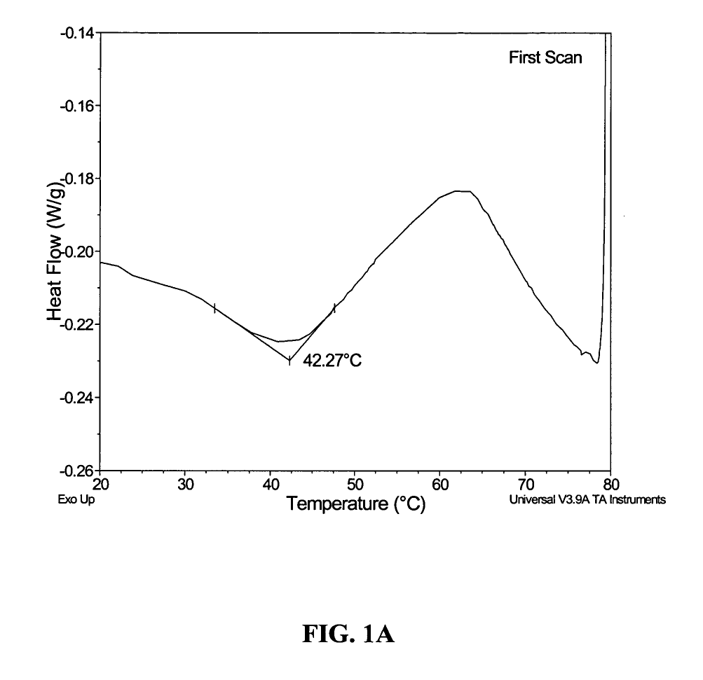

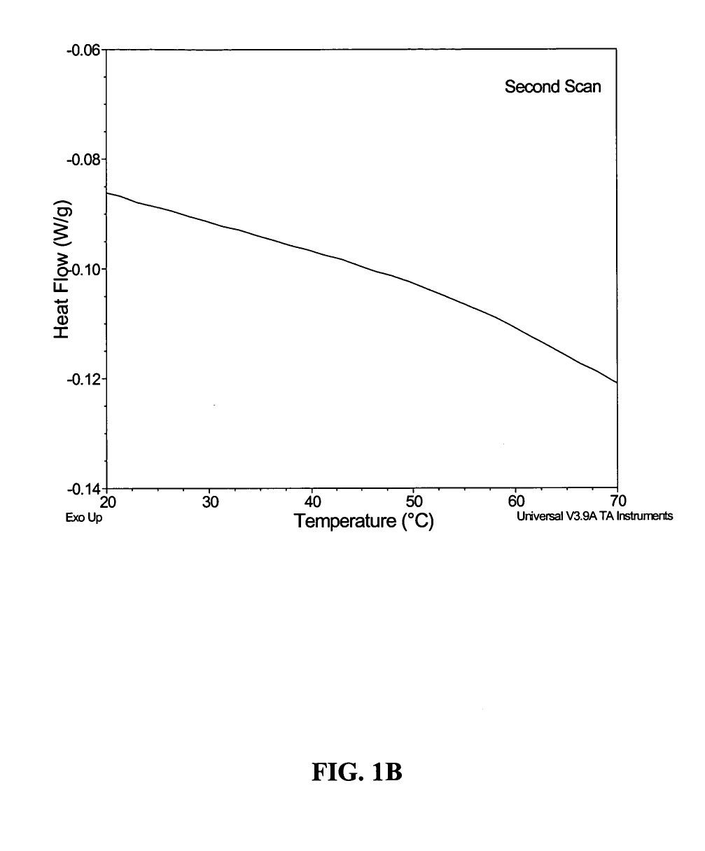

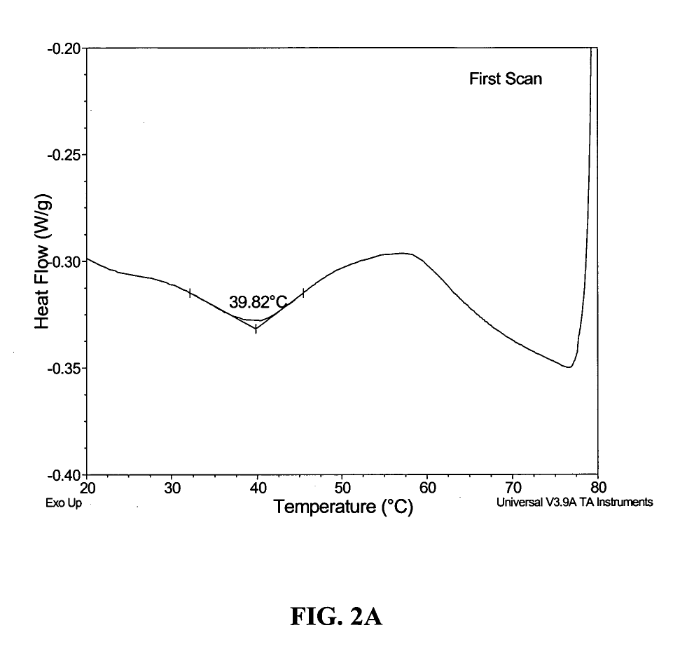

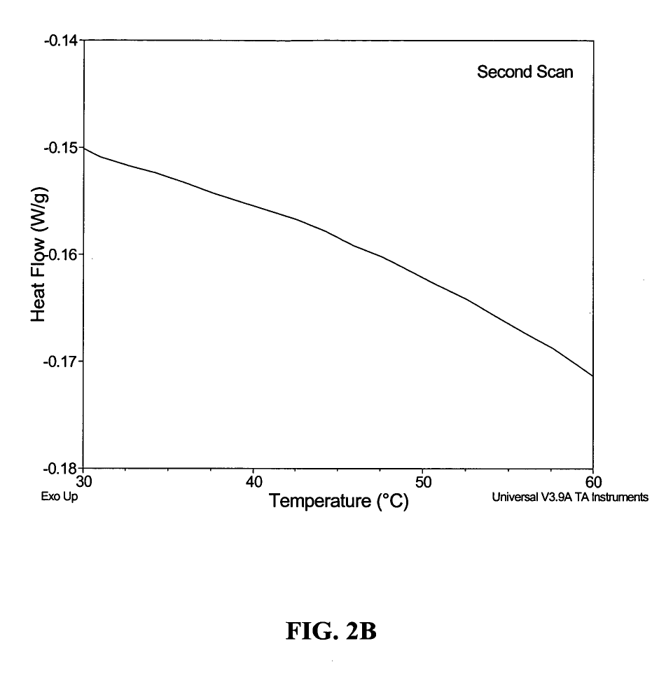

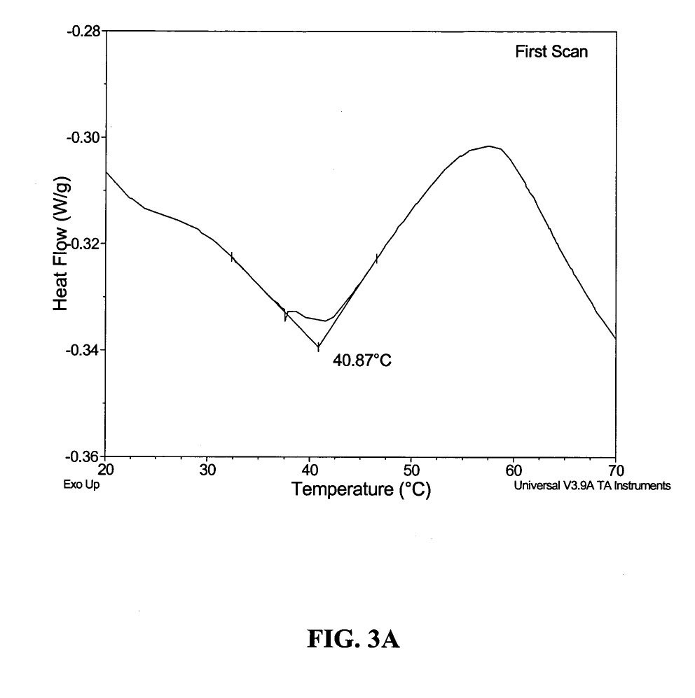

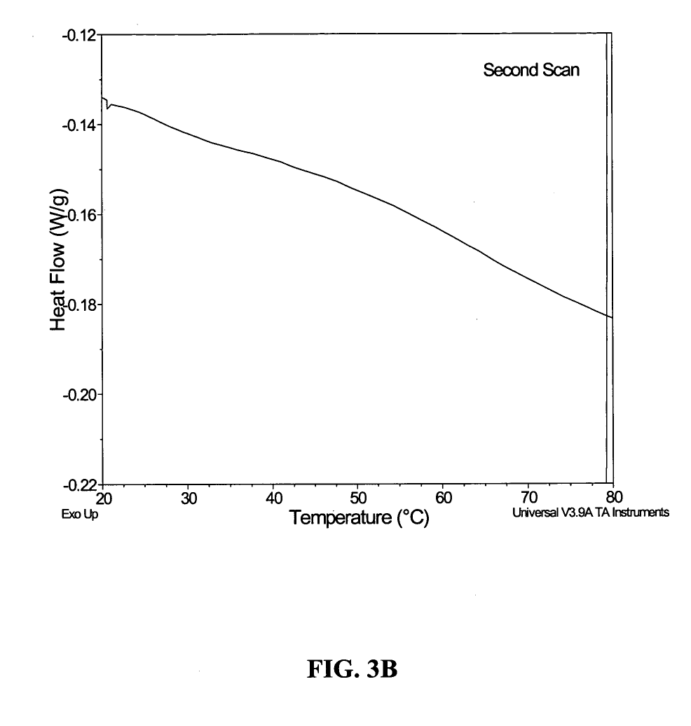

Further, the lyophilized cakes of Formulations 1, 2 and 3 produced by the Lyo G, H and I protocols were assessed for complete crystallization. The assessment for complete crystallization was conducted by DSC. What is observed on the first scan (FIGS. 1A, 2A and 3A) is a non-reversible exothermic event. The absence of the exothermic event on the second scan confirms that it is non-reversible. One skilled in the art recognizes that this non-reversible exothermic event represents a crystallization event. This indicates that a crystallizing excipient did not completely crystallize during lyophilization. FIG. 1A shows a first scan and FIG. 1B shows a second scan on a lyophilized formulation 1, where the scans are representative for Lyo G, H and I. FIG. 2A shows a first scan and FIG. 2B shows a second scan on a lyophilized formulation 2, where the scans are representative for Lyo G, H and I. FIG. 3A shows a first scan and FIG. 3B shows a second scan on a lyophilized formulation 3, where the scans are representative for Lyo G, H and I. As can be observed in FIGS. 1, 2 and 3, all of the lyophilized samples prepared by the Lyo G, H and I protocols showed a crystallization event during the first scan (and no transition during the second scan), indicating that the glycine did not completely crystallize during lyophilization.

### Example 2

### Lyophilization Methods with High Temperature Annealing

In order to provide complete crystallization or improve crystallization of excipients in the cake, lyophilization methods comprising a high-temperature annealing step was tested. An additional aim was to improve the final percent residual moisture to below 1%.

Table 7 shows the formulations used in Example 2. Each formulation contained Factor IX at 250 IU/mL, pH 6.8.

TABLE 7
Formulations Used in Example 2

| Formulation# | NaCl (mM) | Glycine (mM) |
|---|---|---|
| 4 (Control) | 0 | 260 |
| 5 | 50 | 250 |
| 6 | 50 | 270 |

Tables 8, 9 and 10 show the lyophilization steps for Lyo J, K and L. All vials were stoppered under vacuum.

TABLE 8
Lyophilization Cycle “Lyo J”

| Step | Shelf Temperature | Duration | |
|---|---|---|---|
| Freezing Hold | −50° C. | 5 | hours |
| Annealing Hold | −15° C. | 5 | hours |
| Re-Freezing Hold | −50° C. | 5 | hours |
| Primary Drying | 0° C. | 38 | hours |
| Annealing Hold | 50° C. | 3 | hours |
| Secondary Drying | 25° C. | 0 | minutes |

TABLE 9
Lyophilization Cycle “Lyo K”

| Step | Shelf Temperature | Duration |
|---|---|---|
| Freezing Hold | −50° C. | 5 hours |
| Annealing Hold | −15° C. | 5 hours |
| Re-Freezing Hold | −50° C. | 5 hours |
| Primary Drying | 0° C. | 38 hours |
| Annealing Hold | 50° C. | 3 hours |
| Secondary Drying | 25° C. | 9.5 hours |

TABLE 10
Lyophilization Cycle “Lyo L”

| Step | Shelf Temperature | Duration |
|---|---|---|
| Freezing Hold | −50° C. | 5 hours |
| Annealing Hold | −15° C. | 5 hours |
| Re-Freezing Hold | −50° C. | 5 hours |
| Primary Drying | 0° C. | 38 hours |
| Annealing Hold | 50° C. | 10 hours |
| Secondary Drying | 25° C. | 9.5 hours |

Formulations 4, 5, and 6 were each lyophilized in duplicate according to the protocols of Lyo J, Lyo K and Lyo L. The moisture content of each lyophilized product was then assessed using Karl Fisher (see Table 11).

The lyophilized cakes of Formulations 4-6 produced by the Lyo J, K and L cycles were then assessed for complete crystallization. The assessment for complete crystallization was conducted by DSC.

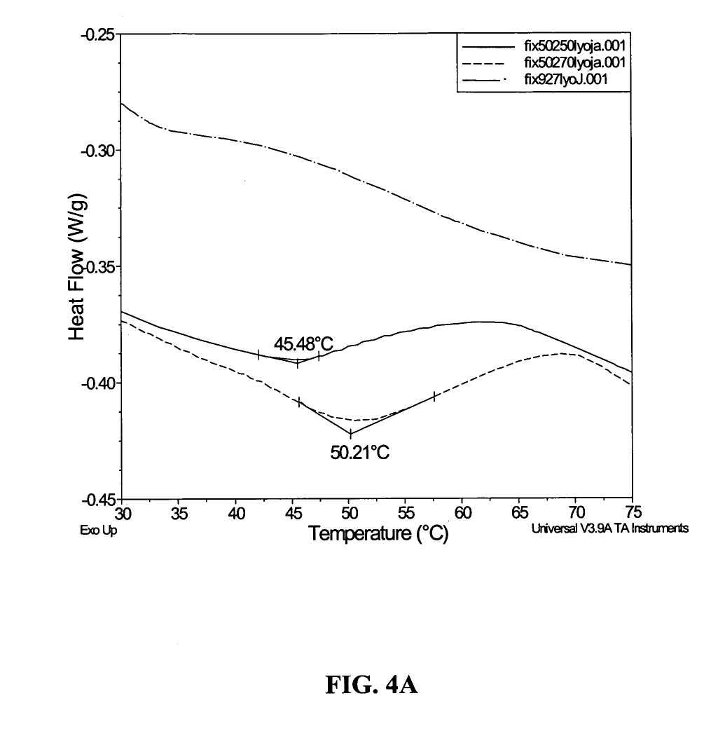

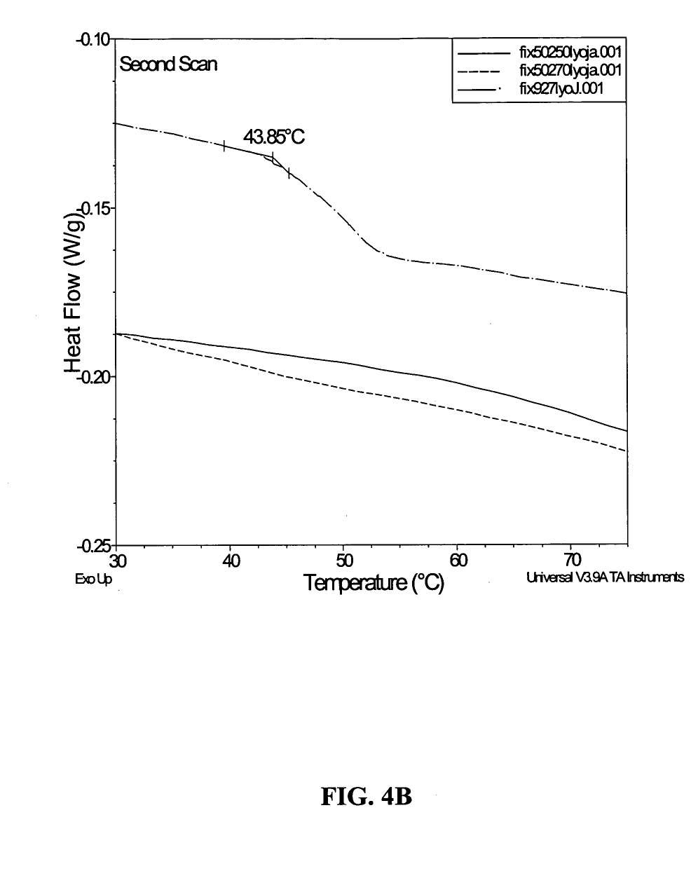

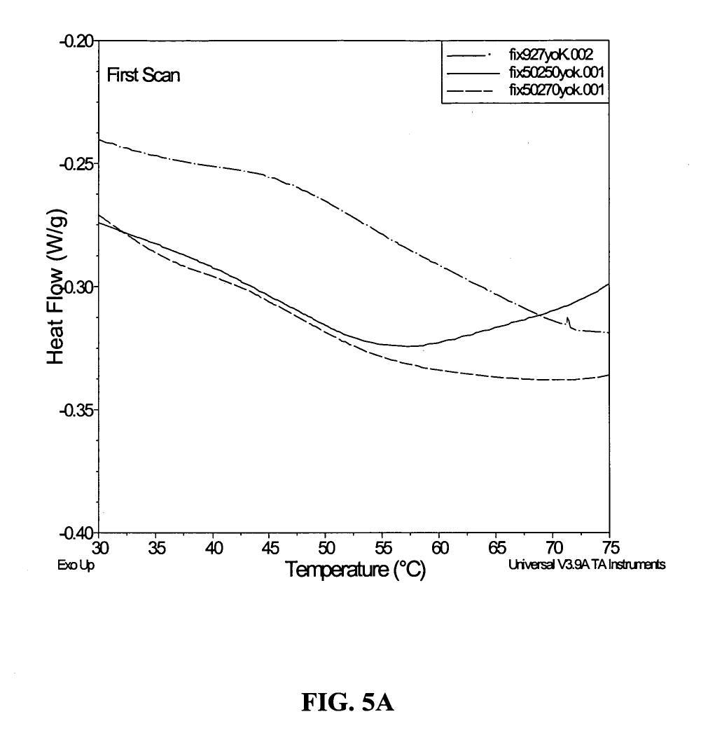

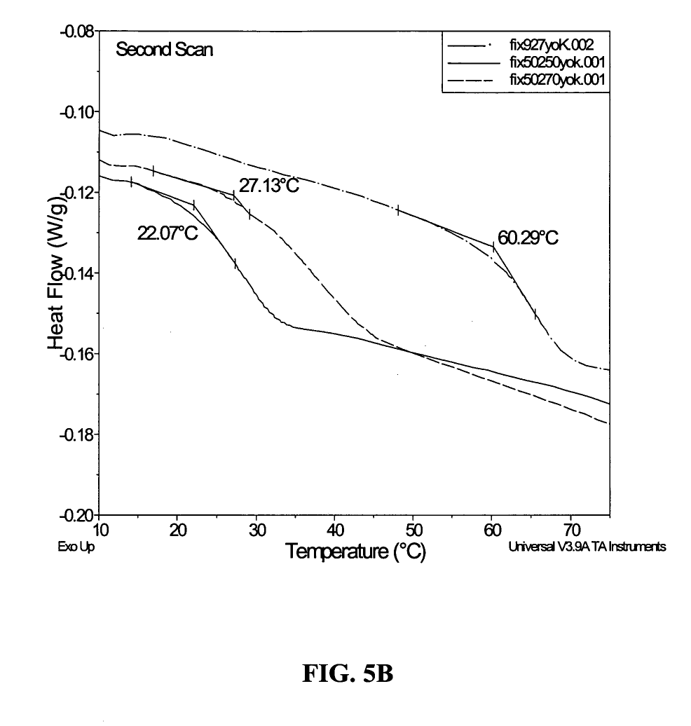

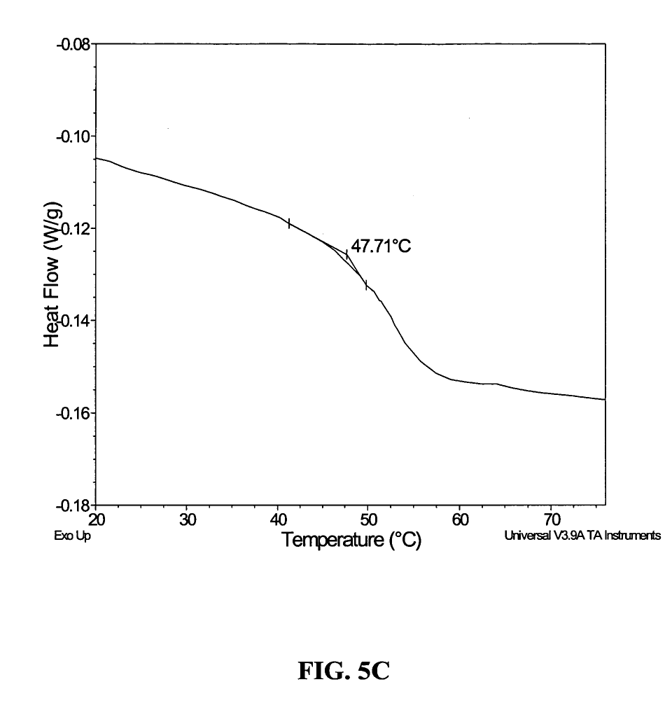

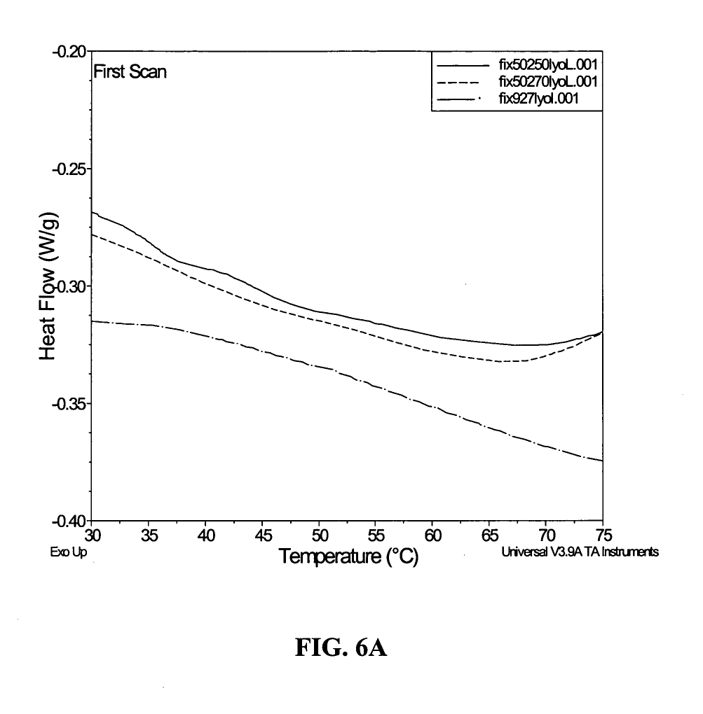

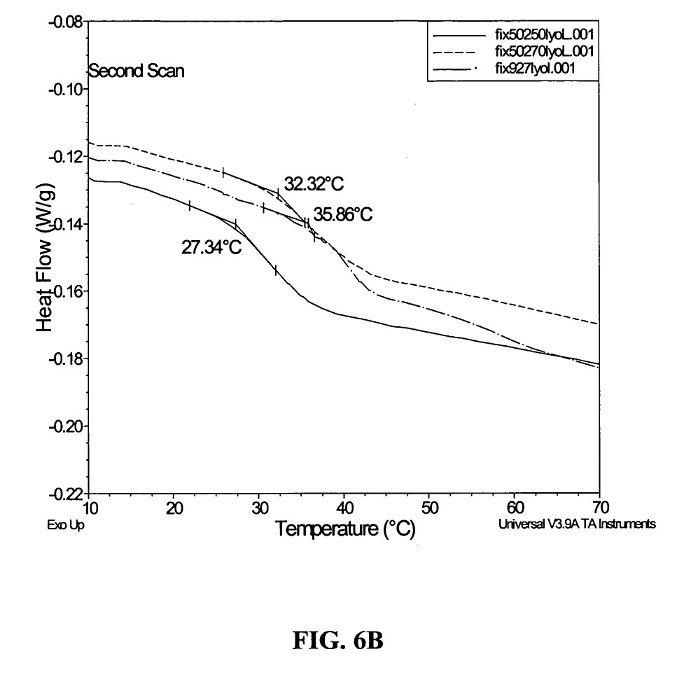

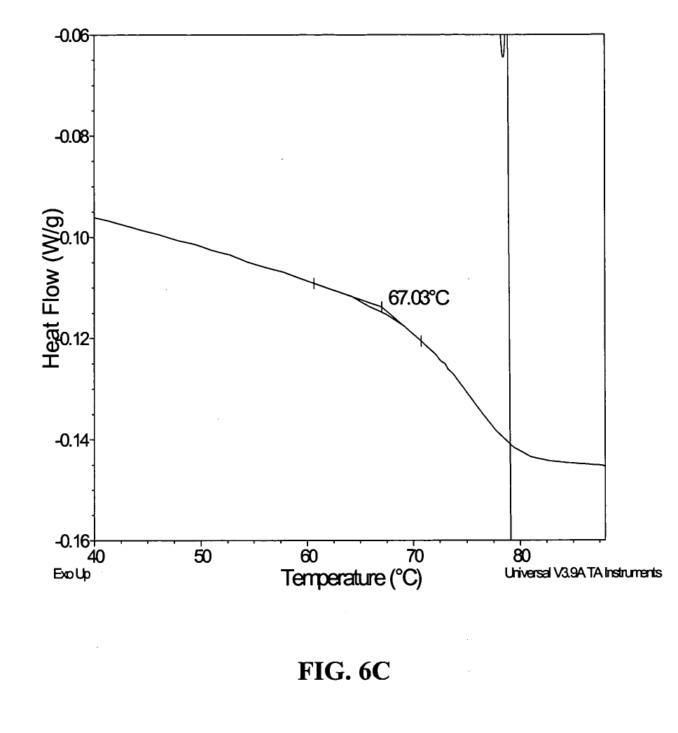

Again, if a crystallization event is observed with the first scan, then the sample did not completely crystallize during lyophilization. FIG. 4A shows a first scan and FIG. 4B shows a second scan on lyophilized formulation 4 (“fix927lyoJ.001”), lyophilized formulation 5 (“fix50250lyoja.001”) and lyophilized formulation 6 (“fix50270lyoja.001”), where the formulations were lyophilized by LyoJ. FIG. 5A shows a first scan and FIG. 5B shows a second scan on lyophilized formulation 4 (“fix927yoK.002”), lyophilized formulation 5 (“fix50250yok.001”) and lyophilized formulation 6 (“fix50270yok.001”), where the formulations were lyophilized according to LyoK. FIG. 5C presents data from another second scan on formulation 4 (“fix 927 lyo K”) lyophilized by the LyoK cycle. FIG. 6A shows a first scan and FIG. 6B shows a second scan on lyophilized formulation 4 (“fix927lyol.001”), lyophilized formulation 5 (“fix50250lyoL.001”) and lyophilized formulation 6 (“fix50270lyoL.001”), where the formulations were lyophilized according to LyoL. FIG. 6C shows another second scan on formulation 4 (“fix 927 Lyo L”) lyophilized according to LyoL.

Table 11 presents a summary of the DSC first and second scan data on formulations 4, 5 and 6 lyophilized according to Lyo J, K or L.

TABLE 11
Summary of % Moisture and DSC First and Second Scan Data for
Example 2

| | | | Crystallization |
|---|---|---|---|
| | | | Event |
| Cycle | Formulation | % Moisture | in First Scan |
| Lyo J | 4 | 0.4 | No |
| | 5 | 1.2 | Yes |
| | 6 | 1.2 | Yes |
| Lyo K | 4 | 0.6 | No |
| | 5 | 1.2 | No |
| | 6 | 1.1 | No |
| Lyo L | 4 | 0.3 | No |
| | 5 | 0.7 | No |
| | 6 | 0.7 | No |

In relation to formulations 5 and 6, Lyo J did not completely crystallize the excipients as a crystallization event was observed with the first scan. Without being bound by theory, this is probably due to the fact that the secondary drying step in Lyo J has a duration of zero hours and that the high-temperature annealing step was only 3 hours. Lyo J was capable of fully crystallizing the excipients in formulation 4, but formulation 4 does not contain a combination of sodium chloride and glycine, whereas formulations 5 and 6 do have a combination of sodium chloride and glycine. Lyo K showed complete crystallization and the residual moisture in the final cakes were less than 1.2%. Lyo L showed superior results, as complete crystallization occurred and as the residual moisture in the final cakes were less than 1%.

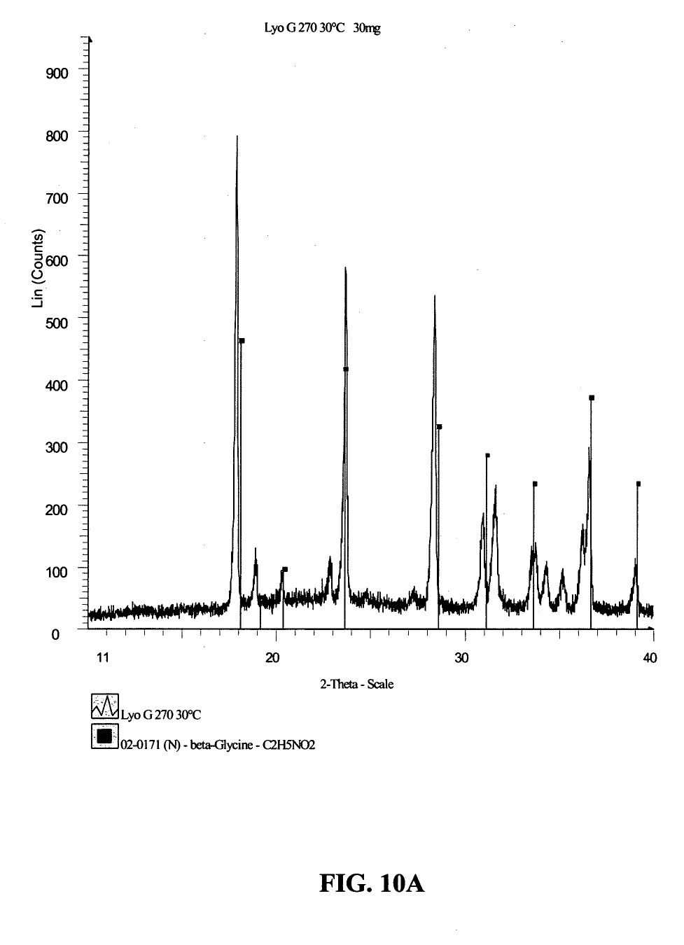

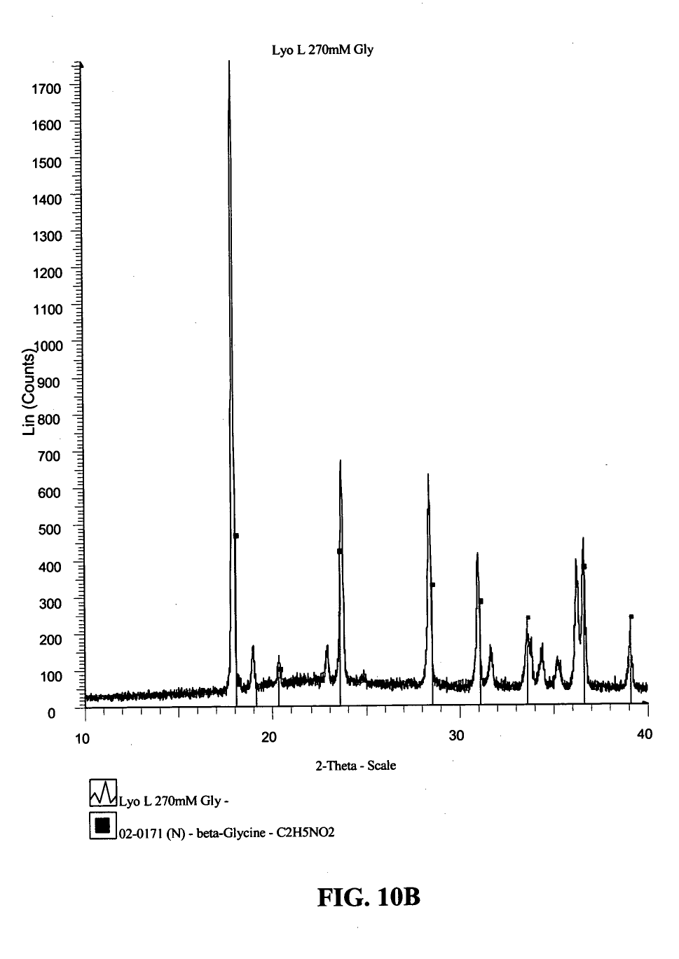

Additionally, three-month stability studies on cakes lyophilized according to the Lyo L cycle show that the moisture content remains less than 1%. Further, X-ray diffraction analysis on samples lyophilized with a high-temperature annealing step showed an increase in glycine crystallization when compared to samples lyophilized without a high temperature (higher than 35° C.) annealing step (see FIG. 10A and FIG. 10B).

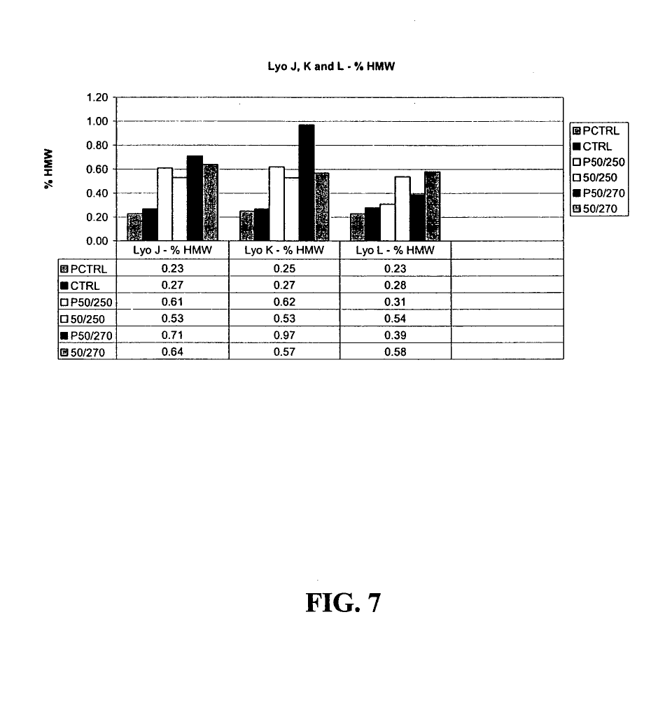

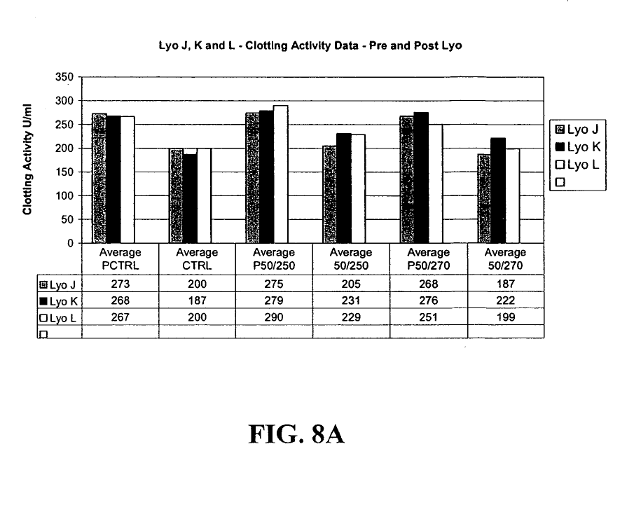

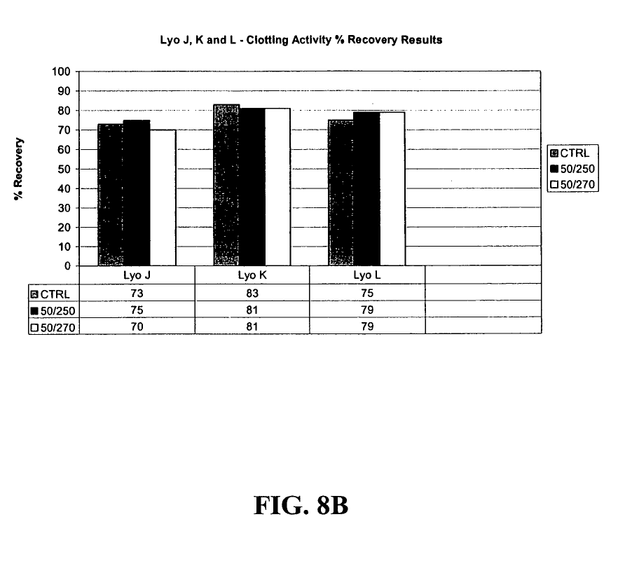

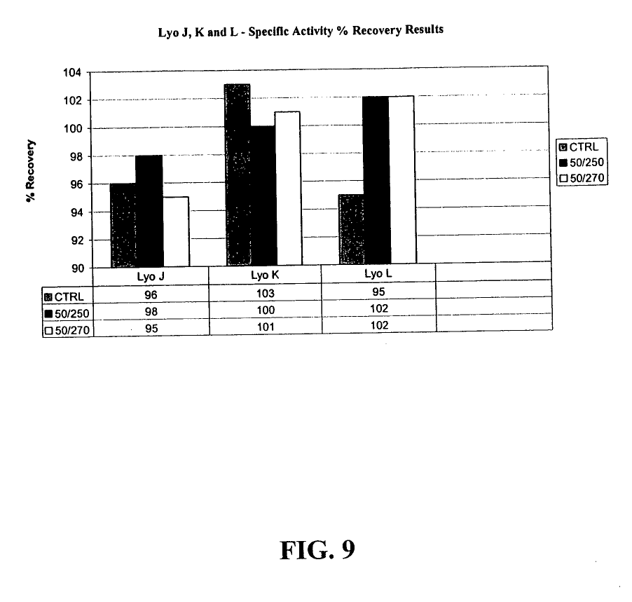

Stability was also tested on the lyophilized products by assaying (1) the percent of high molecular weight (HMW) species present in the final cakes, (2) the percent recovery of clotting activity of Factor IX and (3) the percent recovery of specific activity of Factor IX. The percent high molecular weight is determined by size exclusion chromatography (SEC-HPLC). Clotting activity was determined using a one-stage activated partial thromboplastin time assay. Specific activity was calculated by dividing the clotting activity by the protein concentration, and the protein concentration was determined by using SEC-HPLC. FIG. 7 shows the % HMW for Formulations 4-6 lyophilized by Lyo J, K and L. FIGS. 8A and 8B show the clotting activity data of Factor IX pre and post lyophilization (due to a 4 mL fill and a 5 mL reconstitution dilution, 80% recovery is the highest recovery value expected in FIG. 8B). FIG. 9 shows the specific activity percent recovery. These results indicate that Factor IX lyophilized by methods having high-temperature annealing and drying steps was not negatively affected, as shown by % HMW and by potency assays.

### Example 3

### Lyophilization Methods with High Temperature Annealing Provides Long-Term Stability

The results in Example 2 indicate that formulations were not negatively affected by a thermal treatment (or “Annealing Hold”; see Tables 9 and 10) step at 50° C. In fact, this thermal treatment step resulted in lower percent (%) residual moisture values for formulations comprising sodium chloride and glycine. This reduction in % moisture correlated with an increase in glycine crystallization. To provide further evidence that lyophilization cycles conducted with a high-temperature thermal treatment step does not negatively affect active-ingredient stability, the following experiments were conducted.

Table 12 shows the formulation filled and lyophilized in this Example. The formulation contained recombinant Factor IX at either 69 IU/mL, pH 6.8 or 550 IU/mL, pH 6.8.

TABLE 12
Formulation Used in Example 3

| Formulation # | NaCl (mM) | Glycine (mM) |
|---|---|---|
| 7 | 50 | 260 |

Two lyophilization cycles (Lyo P and Lyo Q) were performed with the only difference between the cycles was that one set was stoppered under full vacuum and the other was stoppered with a nitrogen headspace in the vials. Tables 13 and 14 list the lyophilization cycles for Lyo P and Lyo Q. For Lyo P, all vials were stoppered under vacuum. For Lyo Q, all vials were stoppered with a nitrogen headspace.

TABLE 13
Lyophilization Cycle “Lyo P”

| Step | Shelf Temperature | Duration |
|---|---|---|
| Freezing Hold | −50° C. | 5 hours |
| Annealing Hold | −15° C. | 5 hours |
| Re-Freezing Hold | −50° C. | 5 hours |
| Primary Drying | 0° C. | 38 hours |
| Thermal Treatment | 50° C. | 10 hours |
| Secondary Drying | 25° C. | 9.5 hours |

TABLE 14
Lyophilization Cycle “Lyo Q”

| Step | Shelf Temperature | Duration |
|---|---|---|
| Freezing Hold | −50° C. | 5 hours |
| Annealing Hold | −15° C. | 5 hours |
| Re-Freezing Hold | −50° C. | 5 hours |
| Primary Drying | 0° C. | 38 hours |
| Thermal Treatment | 50° C. | 10 hours |
| Secondary Drying | 25° C. | 9.5 hours |

Multiple cakes (all cake appearances were good) were produced by Lyo P and Lyo Q in order to test residual moisture levels of these cakes in storage at various times and at various temperatures. At all times and temperatures tested, the cakes had residual moisture levels below 1.5% (residual moisture levels below 2% are generally acceptable). Even after 9 months and 12 months at 50° C., the cakes had residual moisture levels below 1%. The results are listed below in Table 15:

TABLE 15
Residual Moisture Levels

| | | 3 months at | 9 months at | 12 months at |
|---|---|---|---|---|
| Sample | Initial | 40° C. | 50° C. | 40° C. |
| Lyo P 69 IU/mL | 0.67 | 1.17 | 0.79 | 0.79 |
| Lyo P 550 IU/mL | 0.54 | 1.12 | 0.84 | 0.83 |
| Lyo Q 69 IU/mL | 0.59 | 1.29 | 0.83 | 0.89 |
| Lyo Q 550 IU/mL | 0.55 | 1.22 | 0.95 | 0.80 |

Stability of cakes produced by Lyo P and Lyo Q were tested by assaying for the percentage of HMW species present in the cakes. These stability tests were conducted where the cakes were stored at 2-8° C. and at higher storage temperatures. Less than 3% HMW is acceptable. Experiments were conducted with a single vial per timepoint, each timepoint was assayed in triplicate using SEC-HPLC. The results are presented below in Tables 16A-D:

TABLE 16A
% HMW Results For Lyo P (69 IU/mL or 250 IU/Vial)

| Storage Temp. | 0 Months | 3 Months | 9 Months | 12 Months |
|---|---|---|---|---|
| −80° C. | 0.51 | 0.35 | 0.55 | 0.57 |
| 2-8° C. | 1.27 | 0.57 | — | 0.94 |
| 25° C. | 1.27 | 0.65 | — | 1.03 |
| 30° C. | 1.27 | 0.63 | — | 0.78 |
| 40° C. | 1.27 | 0.77 | 0.71 | 1.09 |
| 50° C. | 1.27 | 0.84 | 0.82 | 0.81 |

TABLE 16B
% HMW Results For Lyo P (550 IU/mL or 2000 IU/Vial)

| Storage Temp. | 0 Months | 3 Months | 9 Months | 12 Months |
|---|---|---|---|---|
| −80° C. | 0.36 | 0.38 | 0.51 | 0.47 |
| 2-8° C. | 0.54 | 0.41 | — | 0.53 |
| 25° C. | 0.54 | 0.46 | — | 0.59 |
| 30° C. | 0.54 | 0.46 | — | 0.60 |
| 40° C. | 0.54 | 0.51 | 0.61 | 0.74 |
| 50° C. | 0.54 | 0.55 | 0.63 | 0.75 |

TABLE 16C
% HMW Results For Lyo Q (69 IU/mL or 250 IU/Vial)

| Storage Temp. | 0 Months | 3 Months | 9 Months | 12 Months |
|---|---|---|---|---|
| −80° C. | 0.43 | 0.41 | 0.41 | 0.49 |
| 2-8° C. | 1.76 | 0.62 | — | 0.95 |
| 25° C. | 1.76 | 0.59 | — | 0.92 |
| 30° C. | 1.76 | 0.69 | — | 0.91 |
| 40° C. | 1.76 | 0.79 | 0.91 | 1.08 |
| 50° C. | 1.76 | 0.90 | 0.92 | 1.16 |

TABLE 16D
% HMW Results For Lyo Q (550 IU/mL or 2000 IU/Vial)

| Storage Temp. | 0 Months | 3 Months | 9 Months | 12 Months |
|---|---|---|---|---|
| −80° C. | 0.41 | 0.42 | 0.38 | 0.49 |
| 2-8° C. | 0.65 | 0.41 | — | 0.51 |
| 25° C. | 0.65 | 0.46 | — | 0.62 |
| 30° C. | 0.65 | 0.47 | — | 0.58 |
| 40° C. | 0.65 | 0.55 | 0.64 | 0.48 |
| 50° C. | 0.65 | 0.63 | 0.73 | 0.79 |

The stability of drug products lyophilized by Lyo P and Lyo Q was also tested by assaying the concentration of Factor IX. These stability tests were conducted where the cakes were stored at 2-8° C. and at accelerated temperatures. Experiments were conducted with a single vial per timepoint, and each timepoint was assayed in triplicate. The results (in units of μg/mL) are presented below in Tables 17A-D. Note*: the −80° C. time points in Tables 17A-D are controls, where a pre-lyophilized bulk drug product (BDP) control is tested. Four mLs of the BDP were filled into each vial and lyophilized. The resultant vials were reconstituted with 5 mLs of water for injection (WFI). This results in a drug product that is 20% less concentrated than the BDP control. The concentration of Factor IX in the reconstituted vials was determined by SEC-HPLC.

TABLE 17A
Factor LX Protein Concentration Results For Lyo P (69 IU/mL or
250 IU/Vial)

| Storage Temp. | 0 Months | 3 Months | 9 Months | 12 Months |
|---|---|---|---|---|
| −80° C.* | 247 | 240 | 223 | 231 |
| 2-8° C. | 186 | 190 | — | 179 |
| 25° C. | 186 | 191 | — | 179 |
| 30° C. | 186 | 187 | — | 161 |
| 40° C. | 186 | 188 | 171 | 173 |
| 50° C. | 186 | 175 | 169 | 160 |

TABLE 17A
Factor IX Protein Concentration Results For Lyo P (550 IU/mL or
2000 IU/Vial)

| Storage Temp. | 0 Months | 3 Months | 9 Months | 12 Months |
|---|---|---|---|---|
| −80° C.* | 2,524 | 2,593 | 2,323 | 2,322 |
| 2-8° C. | 2,032 | 2,091 | — | 1,788 |
| 25° C. | 2,032 | 2,132 | — | 1,845 |
| 30° C. | 2,032 | 2,135 | — | 1,842 |
| 40° C. | 2,032 | 2,120 | 1,817 | 1,845 |
| 50° C. | 2,032 | 2,109 | 1,770 | 1,825 |

TABLE 17C
Factor IX Protein Concentration Results For Lyo Q
(69 IU/mL or 250 IU/Vial)

| Storage Temp. | 0 Months | 3 Months | 9 Months | 12 Months |
|---|---|---|---|---|
| −80° C.* | 245 | 239 | 227 | 220 |
| 2-8° C. | 178 | 187 | — | 173 |
| 25° C. | 178 | 185 | — | 176 |
| 30° C. | 178 | 185 | — | 175 |
| 40° C. | 178 | 181 | 165 | 169 |
| 50° C. | 178 | 183 | 161 | 168 |

TABLE 17D
Factor IX Protein Concentration Results For Lyo Q (550 IU/mL
or 2000 IU/Vial)

| Storage Temp. | 0 Months | 3 Months | 9 Months | 12 Months |
|---|---|---|---|---|
| −80° C.* | 2,572 | 2,665 | 2,301 | 2,409 |
| 2-8° C. | 1,983 | 2,094 | — | 1,810 |
| 25° C. | 1,983 | 2,094 | — | 1,821 |
| 30° C. | 1,983 | 2,107 | — | 1,819 |
| 40° C. | 1,983 | 2,098 | 1,734 | 1,701 |
| 50° C. | 1,983 | 2,100 | 1,735 | 1,830 |

Stability of cakes produced by Lyo P and Lyo Q was also tested by assaying the potency of clotting activity of Factor IX. These stability tests were conducted where the cakes were stored at 2-8° C. and at accelerated temperatures. Experiments were conducted with a single vial per timepoint, and the results, presented below in Tables 18A-D, are in units of IU/mL. Further, in Tables 18A-D, the −80° C. data points are controls with a pre-lyophilized bulk drug product control (BDP). Four mLs of the BDP are filled into each vial and lyophilized. The resultant vial is reconstituted with 5 mLs of WFI. This results in a drug product that has 20% less potency than the BDP control. Thus, for Table 18A, 44 IU/mL is equivalent to 100% activity for reconstituted BDP (20% less potency than the BDP −80° C. control at 12 months). Similarly, for Table 18B, 593 IU/mL is equivalent to 100% activity for reconstituted BDP; for Table 18C, 49 IU/mL is equivalent to 100% activity for reconstituted BDP; and for Table 18D, 697 IU/mL is equivalent to 100% activity for reconstituted BDP. Factor IX potency was determined by assaying the clotting activity using a one-stage activated partial thromboplastin time assay.

TABLE 18A
Factor IX Potency Results For Lyo P (69 IU/mL or 250 IU/Vial)

| Storage Temp. | 0 Months | 3 Months | 9 Months | 12 Months |
|---|---|---|---|---|
| −80° C.* | 48 | 69 | 64 | 55 |
| 2-8° C. | 37 | 54 | — | 54 |
| 25° C. | 37 | 52 | — | 54 |
| 30° C. | 37 | 48 | — | 51 |
| 40° C. | 37 | 52 | 45 | 46 |
| 50° C. | 37 | 43 | 44 | 48 |

TABLE 18B
Factor IX Potency Results For Lyo P (550 IU/mL or 2000 IU/Vial)

| Storage Temp. | 0 Months | 3 Months | 9 Months | 12 Months |
|---|---|---|---|---|
| −80° C.* | 505 | 739 | 735 | 741 |
| 2-8° C. | 380 | 507 | — | 564 |
| 25° C. | 380 | 426 | — | 587 |
| 30° C. | 380 | 459 | — | 640 |
| 40° C. | 380 | 486 | 511 | 585 |
| 50° C. | 380 | 495 | 578 | 577 |

TABLE 18C
Factor IX Potency Results For Lyo Q (69 IU/mL or 250 IU/Vial)

| Storage Temp. | 0 Months | 3 Months | 9 Months | 12 Months |
|---|---|---|---|---|
| −80° C.* | 44 | 54 | 66 | 61 |
| 2-8° C. | 40 | 40 | — | 57 |
| 25° C. | 40 | 41 | — | 52 |
| 30° C. | 40 | 41 | — | (sample |
| | | | | handling error) |
| 40° C. | 40 | 37 | 38 | 45 |
| 50° C. | 40 | 38 | 39 | 40 |

TABLE 18D
Factor IX Potency Results For Lyo Q (550 IU/mL or 2000 IU/Vial)

| Storage Temp. | 0 Months | 3 Months | 9 Months | 12 Months |
|---|---|---|---|---|
| −80° C.* | 642 | 747 | 759 | 871 |
| 2-8° C. | 472 | 437 | — | 612 |
| 25° C. | 472 | 450 | — | 609 |
| 30° C. | 472 | 459 | — | 624 |
| 40° C. | 472 | 427 | 353 | 595 |
| 50° C. | 472 | 451 | 549 | 521 |

Stability of cakes produced by Lyo P and Lyo Q was also assessed by determining the specific activity of Factor IX. Factor IX specific activity was calculated by dividing the clotting activity by the protein concentration. The data points below are in units of IU/mg.

TABLE 19A
Factor IX Specific Activity Results For Lyo P (69 IU/mL or 250 IU/Vial)

| Storage Temp. | 0 Months | 3 Months | 9 Months | 12 Months |
|---|---|---|---|---|
| −80° C. | 193 | 287 | 287 | 238 |
| 2-8° C. | 200 | 284 | — | 302 |
| 25° C. | 200 | 273 | — | 304 |
| 30° C. | 200 | 256 | — | 315 |
| 40° C. | 200 | 276 | 263 | 265 |
| 50° C. | 200 | 246 | 260 | 300 |

TABLE 19B
Factor IX Specific Activity Results For Lyo P (550 IU/mL or
2000 IU/Vial)

| Storage Temp. | 0 Months | 3 Months | 9 Months | 12 Months |
|---|---|---|---|---|
| −80° C. | 200 | 285 | 316 | 319 |
| 2-8° C. | 187 | 242 | — | 316 |
| 25° C. | 187 | 200 | — | 318 |
| 30° C. | 187 | 215 | — | 347 |
| 40° C. | 187 | 229 | 281 | 317 |
| 50° C. | 187 | 235 | 327 | 316 |

TABLE 19C
Factor IX Specific Activity Results For Lyo Q (69 IU/mL or 250 IU/Vial)

| Storage Temp. | 0 Months | 3 Months | 9 Months | 12 Months |
|---|---|---|---|---|
| −80° C. | 179 | 226 | 291 | 277 |
| 2-8° C. | 222 | 214 | — | 328 |
| 25° C. | 222 | 221 | — | 294 |
| 30° C. | 222 | 222 | — | (sample |
| | | | | handling error) |
| 40° C. | 222 | 204 | 230 | 267 |
| 50° C. | 222 | 207 | 242 | 235 |

TABLE 19D
Factor IX Specific Activity Results For Lyo Q (550 IU/mL
or 2000 IU/Vial)

| Storage Temp. | 0 Months | 3 Months | 9 Months | 12 Months |
|---|---|---|---|---|
| −80° C. | 250 | 280 | 330 | 362 |
| 2-8° C. | 238 | 209 | — | 338 |
| 25° C. | 238 | 215 | — | 334 |
| 30° C. | 238 | 218 | — | 343 |
| 40° C. | 238 | 204 | 204 | 350 |
| 50° C. | 238 | 215 | 316 | 285 |

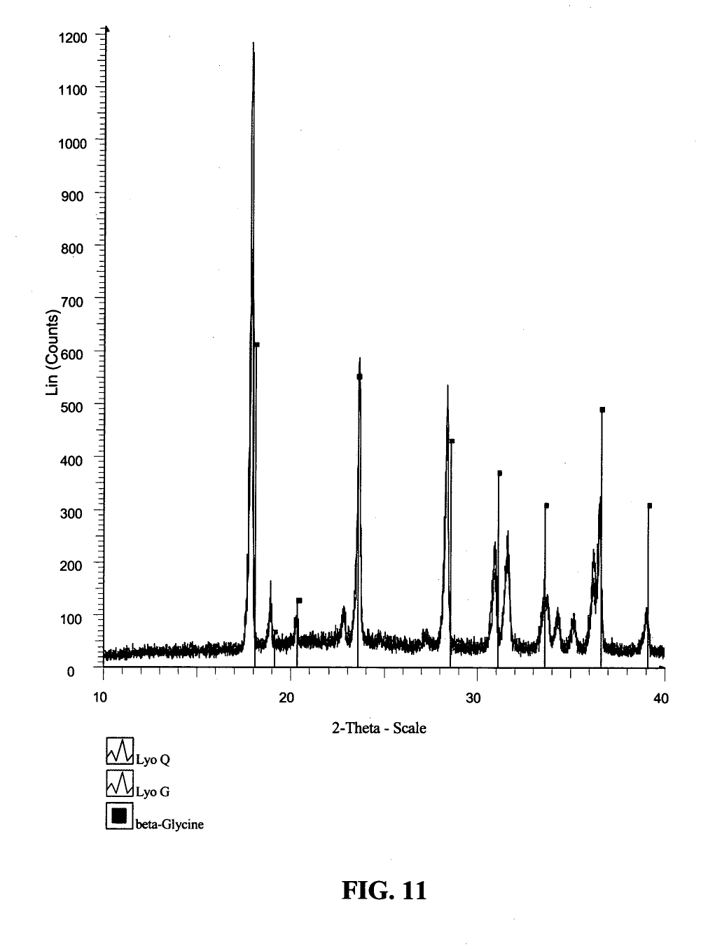

X-Ray diffraction (XRD) was also performed on the lyophilized cakes to observe if the high-temperature thermal treatment step enhanced glycine crystallization (see FIG. 11). XRD was used to identify crystalline structures present in the lyophilized cakes. Lyo G was used for comparison since it included the same lyophilization cycle parameters as Lyo Q, except that Lyo G does not have the 50° C. thermal treatment step prior to secondary drying. FIG. 11 shows that Lyo Q did have an increase in glycine crystallization.

In summary, the results of Example 3 show: (1) the high temperature thermal treatment step did not affect the stability or activity of the active ingredient, Factor IX, as measured by % HMW, potency and specific activity; (2) the stability data shows that Factor IX is stable to the thermal treatment process and is stable over long-term storage at accelerated temperatures; and (3) the high temperature thermal treatment step increased the amount of crystalline glycine. As an added benefit, the high temperature thermal treatment step decreased the final residual moisture value for the samples. Thus, the results of Example 3 provide further evidence that the high-temperature annealing or thermal treatment step prior to secondary drying does not destabilize active ingredients, but rather serves to improve excipient crystallization and overall lyophilization efficiency.

### Example 4

### Low Temperature Annealing Step is Optional

A matrix of lyophilization cycles were performed with and without a −15° C. and 50° C. annealing step to determine if a low temperature annealing step is required to enhance glycine crystallization. Formulation buffer consisting of 10 mM histidine, 1% sucrose, 260 mM glycine, 50 mM NaCl, 0.005% Polysorbate 80, pH 6.8 was used for all cycles. Analysis of the lyophilized cakes consisted of DSC., XRD, and % residual moisture. Table 20 shows the lyophilization cycles performed.

TABLE 20
Lyophilization Cycles

| Step | Shelf Temperature | Duration |
|---|---|---|
| Freezing Hold | −50° C. | 5 hours |
| Annealing Hold | −15° C. | 0 or 5 hours (see Table 21) |
| Re-Freezing Hold | −50° C. | 5 hours |
| Primary Drying | 0° C. | 20 hours |
| Annealing Hold | 50° C. | 0 or 10 hours (see Table 21) |
| Secondary Drying | 25° C. | 9.5 hours |

Table 21 below shows the analytical results of the experiments:

TABLE 21
Results

| | | DSC | | | |
|---|---|---|---|---|---|
| | | | Tg Onset | | |
| | | Crystallization | (° C.) | XRD | |
| Anneal (hours) | | Observed on | Second | 17.5° | % |
| −15° C. | 50° C. | First Scan | Scan | Intensity | Moisture* |
| 0 | 0 | Yes | 31 | 500 | 2.3 |
| 5 | 0 | Yes | 29 | 1450 | 1.9 |
| 0 | 10 | No | 49 | 525 | 0.63 |
| 5 | 10 | No | 52 | 994 | 0.59 |

*Average of 2 vials.

Based on the DSC data, there is evidence that inclusion of the low temperature annealing step (here, −15° C.) does not affect whether a crystallization event is detected on the first scan. The high temperature annealing step (here, 50° C.) is sufficient to eliminate the re-crystallization event as observed on the DSC first scan. Although a lower annealing step is optional, its inclusion is beneficial, because the 17.5° peak from the XRD data suggests that the −15° C. annealing step further enhances the glycine crystallization.

1. A method for lyophilizing an aqueous pharmaceutical formulation, the method comprising:
(a) freezing the aqueous pharmaceutical formulation;
(b) drying the pharmaceutical formulation of step (b);
(c) annealing the pharmaceutical formulation of step (c) at a temperature greater than about 25° C.; and
(d) drying the pharmaceutical formulation of step (c) at a temperature less than the temperature used in step (c).

2. The method of claim 1, wherein the freezing in step (a) is conducted at a temperature of less than −10° C.

3. The method of claim 1, wherein the freezing in step (a) is conducted at a temperature of less than −35° C.

4. The method of claim 1, wherein the drying in step (b) is conducted at a temperature of between about −35° C. and about 20° C.

5. The method of claim 1, wherein the drying in step (b) is conducted at a temperature of between about −25° C. and about 10° C.

6. The method of claim 1, wherein the drying in step (b) is conducted at a temperature of between about −20° C. and about 0° C.

7. The method of claim 1, wherein the drying in step (b) is conducted at a temperature of about 0° C.

8. The method of claim 1, wherein the annealing in step (c) is conducted at a temperature of between about 25° C. and about 75° C.

9. The method of claim 1, wherein the annealing in step (c) is conducted at a temperature of between about 35° C. and about 60° C.

10. The method of claim 1, wherein the annealing in step (c) is conducted at a temperature of about 50° C.

11. The method of claim 1, wherein the drying in step (d) is conducted at a temperature of about 25° C.

12. A method for lyophilizing an aqueous pharmaceutical formulation, the method comprising:
(a) freezing the aqueous pharmaceutical formulation;
(b) annealing the pharmaceutical formulation of step (a) at a temperature between about −35° C. and about 0° C.;
(c) drying the pharmaceutical formulation of step (b) at a temperature between about −35° C. and about 10° C.;
(d) annealing the pharmaceutical formulation of step (c) at a temperature between about 25° C. and about 75° C.; and
(e) drying the pharmaceutical formulation of step (c) at a temperature less than the temperature used in step (d).

13. The method of claim 12, wherein the freezing in step (a) is conducted at a temperature less than −10° C.

14. The method of claim 12, wherein the freezing in step (a) is conducted at a temperature of less than −35° C.

15. The method of claim 12, wherein the annealing in step (b) is conducted at a temperature between about −25° C. and about 10° C.

16. The method of claim 12, wherein the annealing in step (b) is conducted at a temperature between about −20° C. and about −10° C.

17. The method of claim 12, wherein the annealing in step (b) is conducted at a temperature of about −15° C.

18. The method of claim 12, wherein the drying in step (c) is conducted at a temperature of between about −25° C. and about −10° C.

19. The method of claim 12, wherein the drying in step (c) is conducted at a temperature of between about −20° C. and about −10° C.

20. The method of claim 12, wherein the drying in step (c) is conducted at a temperature of about 0° C.

21. The method of claim 12, wherein the annealing in step (d) is conducted at a temperature of between about 35° C. and about 60° C.

22. The method of claim 12, wherein the annealing in step (d) is conducted at a temperature of about 50° C.

23. The method of claim 12, wherein the drying in step (e) is conducted at a temperature of about 25° C.

24. The method of claim 12, further comprising a refreezing step that is conducted after step (b) and prior to step (c), where the refreezing step is conducted at a temperature of less than −35° C.

25. The method of claim 24, wherein the refreezing step is conducted at a temperature between about −40° C. and about −50° C.

26. The method of claim 1 or 12, wherein the aqueous pharmaceutical formulation comprises at least one crystallizing excipient.

27. The method of claim 26, wherein the crystallizing excipient(s) are selected from the group consisting of an amino acid, a salt and a polyol.

28. The method of claim 27, wherein the amino acid is glycine or histidine.

29. The method of claim 27, wherein the salt is sodium chloride.

30. The method of claim 27, wherein the polyol is mannitol.

31. The method of claim 1 or 12, wherein the aqueous pharmaceutical formulation comprises a combination of crystallizing excipients, wherein the combination is a salt and an amino acid.

32. The method of claim 31, wherein the salt is sodium chloride.

33. The method of claim 32, wherein the sodium chloride is present in the formulation at a concentration greater than about 25 mM.

34. The method of claim 32, wherein the sodium chloride is present in the formulation at a concentration between about 25 mM and 200 mM.

35. The method of claim 32, wherein the sodium chloride is present in the formulation at a concentration between about 30 mM and 100 mM.

36. The method of claim 32, wherein the sodium chloride is present in the formulation at a concentration between about 40 mM and 60 mM.

37. The method of claim 32, wherein the sodium chloride is present in the formulation at a concentration of about 50 mM.

38. The method of claim 31, wherein the amino acid is present in the formulation at a concentration between about 1% to about 10%.

39. The method of claim 31, wherein the amino acid is present in the formulation at a concentration between about 1.5% to about 5%.

40. The method of claim 31, wherein the amino acid is present in the formulation at a concentration between about 1.5% to about 3%.

41. The method of claim 31, wherein the amino acid is present in the formulation at a concentration of about 2%.

42. The method of claim 38, 39, 40, 41 or 42, wherein the amino acid is glycine.

43. A method for lyophilizing an aqueous pharmaceutical formulation comprising sodium chloride and glycine, wherein the method comprises:
(a) freezing the aqueous pharmaceutical formulation at a temperature of less than −35° C.;
(b) optionally annealing the pharmaceutical formulation of step (a) at a temperature between about −20° C. and about −10° C.;
(c) drying the pharmaceutical formulation of step (b) at a temperature between about −10° C. and about 10° C.;
(d) annealing the pharmaceutical formulation of step (c) at a temperature between about 35° C. and about 50° C.; and
(e) drying the pharmaceutical formulation of step (d) at a temperature less than the temperature used in step (d).

44. The method of claim 43, wherein the temperature in step (e) is about 25° C.

45. A method for lyophilizing an aqueous pharmaceutical formulation comprising greater than 35mM sodium chloride and between about 250 mM to about 300 mM glycine, wherein the method comprises:
(a) freezing the aqueous pharmaceutical formulation at a temperature of less than −35° C.;
(b) annealing the pharmaceutical formulation of step (a) at about −15° C.;
(c) drying the pharmaceutical formulation of step (b) at about 0° C.;
(d) annealing the pharmaceutical formulation of step (c) at about 50° C.; and (e) drying the pharmaceutical formulation of step (d) at about 25° C.

46. The method of claim 45, further comprising a refreezing step after step (b) and prior to step (c), wherein the refreezing step comprises refreezing the pharmaceutical formulation of step (b) at about −50° C.

47. The method of claim 46, wherein step (a) is conducted for about 5 hours; step (b) is conducted for about 5 hours; step (c) is conducted for about 38 hours; step (d) is conducted for about 5 hours; and step (e) is conducted for about 9.5 hours.

48. A method for increasing excipient crystallization during lyophilization comprising:
(a) providing an aqueous pharmaceutical formulation comprising glycine and sodium chloride;
(b) freezing the aqueous pharmaceutical formulation;
(c) optionally annealing the pharmaceutical formulation of step (b) at a temperature between about −35° C. and about 0° C.;
(d) drying the pharmaceutical formulation of step (b) or step (c) at a temperature between about −35° C. and about 10° C.;
(e) annealing the pharmaceutical formulation of step (d) at a temperature between about 25° C. and about 75° C., such that the glycine is more crystallized after step (e) than before step (e); and
(f) drying the pharmaceutical formulation of step (e) at a temperature that is the same or lower as the temperature used in step (e), thereby increasing excipient crystallization.

49. A lyophilized product produced by a process comprising:
(a) providing a formulation comprising glycine and sodium chloride;
(b) freezing the formulation;
(c) optionally annealing the pharmaceutical formulation of step (b) at a temperature between about −35° C. and about 0° C.;
(d) drying the pharmaceutical formulation of step (b) or step (c) at a temperature between about −35° C. and about 10° C.;
(e) annealing the pharmaceutical formulation of step (d) at a temperature between about 25° C. and about 75° C.; and
(f) drying the pharmaceutical formulation of step (e) at a temperature that is the same or lower as the temperature used in step (e), thereby providing the lyophilized product.

50. The lyophilized product of claim 49, wherein the formulation further comprises an active ingredient.

51. The lyophilized product of claim 50, wherein the active ingredient is a protein, a nucleic acid or a virus.

52. The lyophilized product of claim 50, wherein the active ingredient is Factor IX.

53. The lyophilized product of claim 49, wherein the glycine in the lyophilized product after step (f) is more crystallized than a lyophilized product made by a process without a high-temperature annealing step before secondary drying.

54. A lyophilized product produced by a process comprising:
(a) providing a formulation comprising glycine and sodium chloride;
(b) freezing the formulation;
(c) annealing the formulation of step (b) at a temperature between about −20° C. and about −10° C.;
(d) drying the formulation of step (c) at a temperature between about 0° C. and about 5° C.;
(e) annealing the formulation of step (d) at a temperature between about 35° C. and about 50° C.; and
(f) drying the formulation of step (e) at a temperature that is the same or lower as the temperature used in step (e), thereby providing the lyophilized product.

55. The lyophilized product of claim 54, wherein the glycine in the lyophilized product after step (f) is more crystallized than a lyophilized product made by a process without a high-temperature annealing step before secondary drying.

56. The lyophilized product of claim 54, wherein the lyophilized product is substantially stable for long-terms at high storage temperatures.

57. The lyophilized product of claim 56, wherein the long-term comprises between about 3 months and about 1 year and wherein the accelerated temperature comprises between about 25° C. and about 50° C.
